SEQUENCE LISTING (16 sequences)

SEQ ID NO: 1 - protein, 6 aa, synthetic construct
IEGRMD

SEQ ID NO: 2 - protein, 1252 aa, synthetic construct
SGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 155 aa, Homo sapiens [sequence omitted]

SEQ ID NO: 4 - DNA, 468 bp, Homo sapiens
  source 1..468: mol_type = genomic DNA
atggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 5 - protein, 330 aa, Homo sapiens [sequence omitted]

SEQ ID NO: 6 - DNA, 993 bp, Homo sapiens
  source 1..993: mol_type = genomic DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 7 - protein, 476 aa, Homo sapiens [sequence omitted]

SEQ ID NO: 8 - DNA, 1431 bp, Homo sapiens
  source 1..1431: mol_type = genomic DNA
atggactggacctggaggttcctctttgtggtggcagcagctacaggtgtccagtcccagatgcaggtggtgcagtctggggctgaagtaaagaagcctgggtcctcggtgacggtctcctgcaaggcatctggaggcaccttcagcaactatgctatcagctgggtgcgacaggcccctggacaagggcttgagtggatgggagggatcatccctctttttggtacaccaacctactcacagaacttccagggcagagtcacgattaccgcggacaaatccaccagcacagcccacatggagctgatcagcctgagatctgaggacacggccgtgtattactgtgcgacagatcgctacaggcaggcaaattttgaccgggcccgggttggctggttcgacccctggggccagggcaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 235 aa, Homo sapiens [sequence omitted]

SEQ ID NO: 10 - DNA, 708 bp, Homo sapiens
  source 1..708: mol_type = genomic DNA
atggcctgggctctgctgctcctcaccctcctcactcaggacacagggtcctgggcccagtctgccctgactcagcctgcctccgtgtctgggtctcctggacagtcgatcaccatctcctgcactggaaccaacaatgatgttgggagttataaccttgtctcctggtaccagcagcacccaggcaaagcccccaaaatcatgatttatgaggtcagtaagcggccctcaggggtttctaatcgcttctctggctccaagtctggcaacacggcctccctgacaatctctgggctccaggctgaggacgaggctgattattactgctgctcatatgcaggtagttacactgtggttttcggcggagggaccaaactgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 11 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 12 - protein, 730 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHOURHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 13 - DNA, 2820 bp, Homo sapiens
  source 1..2820: mol_type = genomic DNA
gggagttcagacctagatctttccagttaatcacacaacaaacttagctcatcgcaataaaaagcagctcagagccgactggctcttttaggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaatttaaaatgtcacttacaacaatcctaagacaactactggagagtcatgtttgttgaaattctcattaatgtttatgggtgttttctgttgttttgtttgtcagtgttattttgtcaatgttgaagtgaattaaggtacatgcaagtgtaataacatatctcctgaagatacttgaatggattaaaaaaacacacaggtatatttgctggatgataaagatttcatgggaaaaaaaatcaattaatctgtctaagctgctttctgatgttggtttcttaataatgagtaaaccacaaattaaatgttattttaacctcaccaaaacaatttataccttgtgtccctaaattgtagccctatattaaattatattacatttcaaaaaaaaaaaaaaaa

SEQ ID NO: 14 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 15 - DNA, 1239 bp, Homo sapiens
  source 1..1239: mol_type = genomic DNA
ctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 16 - protein, 6 aa, synthetic construct
HHHHHH

(19) United States
(12) Patent Application Publication
(10) Pub. No.: US 2024/0247038 A1
(43) Pub. Date: Jul. 25, 2024

(54) ISOLATED PROTEIN COMPLEXES AND COMPOSITIONS AND METHODS OF USE THEREOF

(71) Applicant: The University of Vermont and State Agriculture College, Burlington, VT (US)

(72) Inventors: Jeffrey L. SPEES, Burlington, VT (US); Benjamin LIEBMAN, Burlington, VT (US)

(73) Assignee: The University of Vermont and State Agriculture College, Burlington, VT (US)

(21) Appl. No.: 18/431,592

(22) Filed: Feb. 2, 2024

Related U.S. Application Data
(63) Continuation of application No. PCT/US2022/074419, filed on Aug. 2, 2022.
(60) Provisional application No. 63/229,138, filed on Aug. 4, 2021.
(60) Provisional application No. 63/303,408, filed on Jan. 26, 2022.

(51) Int. Cl. C07K 14/50; A61K 38/00; A61P 9/10; C07K 14/705

(52) U.S. Cl. CPC C07K 14/50; A61P 9/10; C07K 14/70596; A61K 38/00; C07K 2319/30

(57) ABSTRACT

The invention features isolated protein complexes and compositions and methods for use thereof. In embodiments, the isolated protein complexes are used for treating conditions associated with reperfusion injury, hypofusion, and/or low/no-reflow. The isolated protein complexes comprise a fusion protein complexed with basic fibroblast growth factor (FGF2), hepatocyte growth factor (HGF), or vascular endothelial growth factor (VEGF). The fusion protein contains an immunoglobulin G (IgG) Fc domain fused to a polypeptide (e.g., Jagged1, a growth factor, or a cytokine).

### CROSS-REFERENCE TO RELATED APPLICATIONS

This application is a continuation under 35 U.S.C. § 111(a) of PCT International Patent Application No. PCT/US2022/074419, filed Aug. 2, 2022, designating the United States and published in English, which claims priority to and the benefit of U.S. Provisional Application No. 63/229,138, filed Aug. 4, 2021 and U.S. Provisional Application No. 63/303,408, filed Jan. 26, 2022, the entire contents of each of which are incorporated by reference herein.

### SEQUENCE LISTING

The present application contains a Sequence Listing which has been submitted electronically in XML format. The entire contents of the electronic XML Sequence Listing, (Date of creation: Sep. 16, 2022; Size: 27,245 bytes; Name: 167914-011901PCT_SL.xml), is incorporated by reference herein.

### STATEMENT OF RIGHTS TO INVENTIONS MADE UNDER FEDERALLY SPONSORED RESEARCH

This invention was made with government support under Grant No. HL132264 awarded by the National Institutes of Health. The government has certain rights in the invention.

### BACKGROUND OF THE INVENTION

Coronary heart disease leading to acute myocardial infarction (MI; heart attack) is a principal cause of mortality worldwide. Cornerstone treatments for MI are designed to restore blood flow (i.e. “reperfuse”) blocked coronary arteries. Percutaneous coronary intervention (PCI) involving angioplasty and stent placement is a standard of care treatment to restore blood circulation in the heart after myocardial infarction (MI, heart attack) caused by a thrombus (vascular blood clot). There are about 1 million PCI procedures performed annually in the United States and over 3 million worldwide. Thrombolytics, which enzymatically break down blood clots, are also used to restore blood circulation after MI. Despite reduced times to intervention, and successful stent placement, 30-50% of primary PCI patients exhibit low- or “no-reflow”, a phenomenon linked to poor outcomes, increased probability of heart failure, and death. Low/no-reflow occurs when macroscopic vessels are opened by stenting or thrombolysis, but distal myocardial perfusion remains compromised. The distal myocardial perfusion can be compromised by capillary plugging and microemboli, vascular leak, edema, swelling of the capillary bed, vasospasm, and/or necrotic breakdown/rupture of the microvasculature (alternatively referred to as “vascular rhexis”). All patients undergoing PCI have reperfusion injury that negatively affects their prognosis.

Vascular damage and dysfunction linked to reperfusion injury and no-reflow result in loss of oxygen and nutrients to cardiac myocytes and progressive myocardial necrosis (i.e. death of cardiac tissue). For MI patients, finding new strategies to deal with reperfusion injury and low/no-reflow are a high priority because half of final infarct size is attributed to cardiac tissue damage that occurs post-PCI. Unfortunately, all recent phase III trials to treat low/no-reflow have failed their clinical endpoints and none have improved long-term patient outcomes. To date, there is no standard of care or FDA-approved drug to treat low/no-reflow after MI.

Thus, there is a present need for improved methods and compositions for treating conditions associated with reperfusion injury and/or low/no-reflow.

### SUMMARY OF THE INVENTION

As described below, the present invention features isolated protein complexes and compositions and methods for use thereof. In embodiments, the isolated protein complexes are used for treating conditions associated with reperfusion injury, hypofusion, and/or low/no-reflow. The isolated protein complexes comprise a fusion protein complexed with basic fibroblast growth factor (FGF2), hepatocyte growth factor (HGF), or vascular endothelial growth factor (VEGF). The fusion protein contains an immunoglobulin G (IgG) Fc domain fused to a polypeptide (e.g., Jagged1, a growth factor, or a cytokine). In embodiments, reperfusion injury, hypofusion, ischemic injury, and/or low/no-reflow is associated with a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like. In embodiments, the hypofusion is cerebral hypofusion, tissue hypofusion, and/or organ hypofusion.

In one aspect, the invention features an isolated complex containing a growth factor polypeptide, or a fragment thereof, and a fusion protein. The fusion protein contains an immunoglobulin G (IgG) Fc polypeptide, or a fragment thereof, and a polypeptide of interest, or a fragment thereof.

In another aspect, the invention features a composition containing the complex of any of the aspects of the invention delineated herein, or embodiments thereof.

In another aspect, the invention features a pharmaceutical composition for increasing vascular integrity, promoting angiogenesis, increasing myocardial salvage, reducing infarct size, and/or preserving cardiac tissue. The composition contains the complex of any of the aspects of the invention delineated herein and a pharmaceutically acceptable excipient.

In another aspect, the invention features a method for producing a complex. The method involves contacting an isolated growth factor polypeptide or a fragment thereof with a fusion protein containing an immunoglobulin G (IgG) Fc polypeptide, or a fragment thereof, and a polypeptide of interest, or a fragment thereof, thereby forming the complex.

In another aspect, the invention features a method for reducing cell damage or cell death following an ischemic event with reperfusion. The method involves contacting a cell with the complex of any of the aspects of the invention delineated herein, or embodiments thereof, thereby reducing cell damage or cell death following the ischemic event with reperfusion.

In another aspect, the invention features a method for increasing vascular integrity, promoting angiogenesis, increasing myocardial salvage, reducing infarct size, and/or preserving tissue in a subject following an ischemic event with reperfusion. The method involves administering to the subject the complex of any of the aspects of the invention delineated herein, or embodiments thereof, thereby increasing vascular integrity, promoting angiogenesis, increasing myocardial salvage, reducing infarct size, and/or preserving cardiac tissue relative to a reference.

In another aspect, the invention features a method for reducing vascular permeability in a subject following an ischemic event with reperfusion. The method involves administering to the subject the complex of any of the aspects of the invention delineated herein, or embodiments thereof, thereby reducing vascular permeability relative to a reference.

In another aspect, the invention features an isolated complex containing fibroblast growth factor (FGF2), or a fragment thereof, and a fusion protein containing an immunoglobulin G (IgG) Fc polypeptide, or a fragment thereof, and a Jagged-1 polypeptide, or a fragment thereof.

In any of the aspects of the invention delineated herein, or embodiments thereof, the growth factor polypeptide is selected from one or more of fibroblast growth factor (FGF2), hepatocyte growth factor (HGF), and vascular endothelial growth factor (VEGF).

In any of the aspects of the invention delineated herein, or embodiments thereof, the polypeptide of interest is a growth factor or a cytokine. In any of the aspects of the invention delineated herein, or embodiments thereof, the polypeptide of interest is Jagged-1.

In any of the aspects of the invention delineated herein, or embodiments thereof, the Fc polypeptide is separated from the polypeptide of interest by a peptide linker. In any of the aspects of the invention delineated herein, or embodiments thereof, the peptide linker contains the amino acid sequence IEGRMD (SEQ ID NO: 1).

In any of the aspects of the invention delineated herein, or embodiments thereof, polypeptides are complexed by only non-covalent interactions. In any of the aspects of the invention delineated herein, or embodiments thereof, the complex does not contain an antibody-antigen interaction. In any of the aspects of the invention delineated herein, or embodiments thereof, the complex does not contain an antibody-antigen interaction and the polypeptides of the complex are associated with one another by only non-covalent interactions.

In any of the aspects of the invention delineated herein, or embodiments thereof, the isolated growth factor polypeptide or a fragment thereof is contacted with the fusion protein at a molar ratio of about 1:1.

In any of the aspects of the invention delineated herein, or embodiments thereof, the ischemic event is associated with reperfusion injury, hypofusion, ischemic injury, and/or no/low-reflow. In embodiments, the hypofusion is tissue and/or organ hypofusion. In embodiments, the hypofusion is cerebral hypofusion.

In any of the aspects of the invention delineated herein, or embodiments thereof, the ischemic event is associated with a burn, diabetic retinopathy, grafted and/or bioengineered tissues, ischemic stroke, ischemic injury, myocardial infarction, organ injury, peripheral artery disease (PAD), sepsis-induced vascular injury, surgery, vascular injury, and/or a wound. In any of the aspects of the invention delineated herein, or embodiments thereof, the ischemic event is associated with a myocardial infarction. In embodiments, the surgery is an organ transplantation.

In any of the aspects of the invention delineated herein, or embodiments thereof, cell death occurs during hypoxia associated with the ischemic event.

In any of the aspects of the invention delineated herein, or embodiments thereof, the contacting occurs within 72 hours of the ischemic event.

In any of the aspects of the invention delineated herein, or embodiments thereof, the cell is an endothelial cell, smooth muscle cell, fibroblast, cardiac myocyte, skeletal muscle cell, peripheral neuron, CNS neuron, astrocyte, oligodendrocyte, pulmonary epithelial cell, liver epithelial cell, or kidney epithelial cell. In any of the aspects of the invention delineated herein, or embodiments thereof, the cell is a vascular endothelial cell, a vascular smooth muscle cell, a vascular or cardiac fibroblast, or a cardiac myocyte. In embodiments, the vascular endothelial cell is a microvascular endothelial cell. In any of the aspects of the invention delineated herein, or embodiments thereof, the cell is a mammalian cell. In any of the aspects of the invention delineated herein, or embodiments thereof, the cell is a human cell. In any of the aspects of the invention delineated herein, or embodiments thereof, the cells are in a subject.

In any of the aspects of the invention delineated herein, or embodiments thereof, the administration is associated with a reduction in vascular permeability relative to a reference. In any of the aspects of the invention delineated herein, or embodiments thereof, the administration is associated with an increases in vascular integrity. In any of the aspects of the invention delineated herein, or embodiments thereof, the administration is associated with a reduction in death of cells. In any of the aspects of the invention delineated herein, or embodiments thereof, the cells contain an endothelial cell, microglial cell, blood-derived cell, smooth muscle cell, fibroblast, cardiac myocyte, skeletal muscle cell, peripheral neuron, CNS neuron, astrocyte, oligodendrocyte, pulmonary epithelial cell, liver epithelial cell, or kidney epithelial cell.

In any of the aspects of the invention delineated herein, or embodiments thereof, the fusion protein has an amino acid sequence with at least 85% identity to the following sequence or a fragment thereof:

(SEQ ID NO: 2)
SGQFELEILSMQNVNGELQNGNCCGGARNPGDRKCTRDECDTYFKVCLKEYQSRVTAGGPCSFG
SGSTPVIGGNTFNLKASRGNDRNRIVLPFSFAWPRSYTLLVEAWDSSNDTVQPDSIIEKASHSG
MINPSRQWQTLKQNTGVAHFEYQIRVTCDDYYYGFGCNKFCRPRDDFFGHYACDQNGNKTCMEG
WMGPECNRAICRQGCSPKHGSCKLPGDCRCQYGWQGLYCDKCIPHPGCVHGICNEPWQCLCETN
WGGQLCDKDLNYCGTHQPCLNGGTCSNTGPDKYQCSCPEGYSGPNCEIAEHACLSDPCHNRGSC
KETSLGFECECSPGWTGPTCSTNIDDCSPNNCSHGGTCQDLVNGFKCVCPPQWTGKTCQLDANE
CEAKPCVNAKSCKNLIASYYCDCLPGWMGQNCDININDCLGQCQNDASCRDLVNGYRCICPPGY
AGDHCERDIDECASNPCLNGGHCQNEINRFQCLCPTGFSGNLCQLDIDYCEPNPCQNGAQCYNR
ASDYFCKCPEDYEGKNCSHLKDHCRTTPCEVIDSCTVAMASNDTPEGVRYISSNVCGPHGKCKS
QSGGKFTCDCNKGFTGTYCHENINDCESNPCRNGGTCIDGVNSYKCICSDGWEGAYCETNINDC
SQNPCHNGGTCRDLVNDFYCDCKNGWKGKTCHSRDSQCDEATCNNGGTCYDEGDAFKCMCPGGW
EGTTCNIARNSSCLPNPCHNGGTCVVNGESFTCVCKEGWEGPICAQNTNDCSPHPCYNSGTCVD
GDNWYRCECAPGFAGPDCRININECQSSPCAFGATCVDEINGYRCVCPPGHSGAKCQEVSGRPC
ITMGSVIPDGAKWDDDCNTCQCLNGRIACSKVWCGPRPCLLHKGHSECPSGQSCIPILDDQCFV
HPCTGVGECRSSSLQPVKTKCTSDSYYQDNCANITFTFNKEMMSPGLTTEHICSELRNLNILKN
VSAEYSIYIACEPSPSANNEIHVAISAEDIRDDGNPIKEITDKIIDLVSKRDGNSIEGRMDPKS
CDKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK.

The invention provides isolated protein complexes and compositions and methods for use thereof. In embodiments, the isolated protein complexes are used for treating a condition associated with reperfusion injury, hypofusion, ischemic injury, and/or low/no-reflow. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The terms “complex,” “complex of polypeptides,” “protein complex,” or “polypeptide complex” is meant a group of two or more associated polypeptides or fragments thereof. A complex is formed, for example, by two polypeptides that bind, interact or otherwise share some mutual affinity (e.g., electrostatic, hydrophobic, ionic, etc.). In embodiments, the polypeptides are non-covalently associated with one another. In particular embodiments, fibroblast growth factor (FGF2), hepatocyte growth factor (HGF), and/or vascular endothelial growth factor (VEGF), and/or fragments thereof interact with an Fc domain of an IgG or a fragment thereof. In embodiments, the term complex as used herein does not encompass antibody/antigen interactions among IgG and FGF2, HGF, or VEGF. In fact, in some embodiments, antibody/antigen interactions are expressly excluded from the term “complex.”

By “18 kd basic fibroblast growth factor (FGF2) polypeptide” or “basic fibroblast growth factor (FGF2) polypeptide” is meant a polypeptide or fragment thereof comprising an amino acid sequence with at least 85% amino acid sequence identity to GenBank Accession No. AAA52533.1, which is reproduced below, and has mitogenic, angiogenic, and/or neurotrophic activity. An exemplary FGF2 polypeptide is provided below.

(SEQ ID NO: 3)
MAAGSITTLPALPEDGGSGAFPPGHFKDPKRLYCKNGGFFLRIHP
DGRVDGVREKSDPHIKLQLQAEERGVVSIKGVCANRYLAMKEDGR
LLASKCVTDECFFFERLESNNYNTYRSRKYTSWYVALKRTGQYKL
GSKTGPGQKAILFLPMSAKS.

By “18 kd basic fibroblast growth factor (FGF2) polynucleotide” or “basic fibroblast growth factor (FGF2) polynucleotide” is meant a polynucleotide or fragment thereof encoding FGF2. An exemplary FGF2 polynucleotide is provided at GenBank Accession No. J04513.1, which is reproduced below.

(SEQ ID NO: 4)
ATGGCAGCCGGGAGCATCACCACGCTGCCCGCCTTGCCCGAGGAT
GGCGGCAGCGGCGCCTTCCCGCCCGGCCACTTCAAGGACCCCAAG
CGGCTGTACTGCAAAAACGGGGGCTTCTTCCTGCGCATCCACCCC
GACGGCCGAGTTGACGGGGTCCGGGAGAAGAGCGACCCTCACATC
AAGCTACAACTTCAAGCAGAAGAGAGAGGAGTTGTGTCTATCAAA
GGAGTGTGTGCTAACCGTTACCTGGCTATGAAGGAAGATGGAAGA
TTACTGGCTTCTAAATGTGTTACGGATGAGTGTTTCTTTTTTGAA
CGATTGGAATCTAATAACTACAATACTTACCGGTCAAGGAAATAC
ACCAGTTGGTATGTGGCACTGAAACGAACTGGGCAGTATAAACTT
GGATCCAAAACAGGACCTGGGCAGAAAGCTATACTTTTTCTTCCA
ATGTCTGCTAAGAGCTGA.

By “immunoglobulin G (IgG) Fc polypeptide” or “IgG1 Fc polypeptide” is meant a polypeptide or fragment thereof comprising an amino acid sequence with at least 85% amino acid sequence identity to UniProtKB/Swiss-Prot Accession No. P01857.1, which is reproduced below, and capable of binding FGF2, HGF, and/or VEGF. An exemplary IgG Fc polypeptide sequence is provided below.

(SEQ ID NO: 5)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK.

By “immunoglobulin G (IgG) Fc polynucleotide” or “IgG1 Fc polynucleotide” is meant a polynucleotide or fragment thereof encoding IgG Fc. An exemplary IgG Fc polynucleotide sequence is provided below (see GenBank Accession No. M87789.1):

(SEQ ID NO: 6)
GCCTCCACCAAGGGCCCATCGGTCTTCCCCCTGGCACCCTCCTCC
AAGAGCACCTCTGGGGGCACAGCGGCCCTGGGCTGCCTGGTCAAG
GACTACTTCCCCGAACCGGTGACGGTGTCGTGGAACTCAGGCGCC
CTGACCAGCGGCGTGCACACCTTCCCGGCTGTCCTACAGTCCTCA
GGACTCTACTCCCTCAGCAGCGTGGTGACCGTGCCCTCCAGCAGC
TTGGGCACCCAGACCTACATCTGCAACGTGAATCACAAGCCCAGC
AACACCAAGGTGGACAAGAAAGTTGAGCCCAAATCTTGTGACAAA
ACTCACACATGCCCACCGTGCCCAGCACCTGAACTCCTGGGGGGA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCAAGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAGCCG
GAGAACAACTACAAGACCACGCCTCCCGTGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGTAAA
TGA.

By “Immunoglobulin G (IgG) polypeptide” is meant an antibody or fragment thereof comprising a heavy chain polypeptide with at least about 85% amino acid sequence identity to GenBank Accession No. AAA02914.1, which is reproduced below, and capable of forming a complex with HGF, FGF2, and/or VEFGA. An exemplary full-length human IgG heavy chain polypeptide is provided below, where the IgG Fc domain is in bold text.

(SEQ ID NO: 7)
MDWTWRFLFVVAAATGVQSQMQVVQSGAEVKKPGSSVTVSCKASG
GTFSNYAISWVRQAPGQGLEWMGGIIPLFGTPTYSQNFQGRVTIT
ADKSTSTAHMELISLRSEDTAVYYCATDRYRQANFDRARVGWFDP
WGQGTLVTVSSASTKGPSVFPLAPSSKSTSGGTAALGCLVKDYFP
EPVTVSWNSGALTSGVHTFPAVLQSSGLYSLSSVVTVPSSSLGTQ
TYICNVNHKPSNTKVDKKVEPKSCDKTHTCPPCPAPELLGGPSVF
LFPPKPKDTLMISRTPEVTCVVVDVSHEDPEVKFNWYVDGVEVHN
AKTKPREEQYNSTYRVVSVLTVLHQDWLNGKEYKCKVSNKALPAP
IEKTISKAKGQPREPQVYTLPPSRDELTKNQVSLTCLVKGFYPSD
IAVEWESNGQPENNYKTTPPVLDSDGSFFLYSKLTVDKSRWQQGN
VFSCSVMHEALHNHYTQKSLSLSPGK.

By “Immunoglobulin G (IgG) polynucleotide” is meant a nucleic acid molecule encoding an IgG polypeptide or fragment thereof. An exemplary polynucleotide encoding a human IgG heavy chain polypeptide is provided at GenBank Accession No. M87789.1, which is reproduced below, where the sequence encoding the IgG Fc domain is in bold text.

(SEQ ID NO: 8)
ATGGACTGGACCTGGAGGTTCCTCTTTGTGGTGGCAGCAGCTACA
GGTGTCCAGTCCCAGATGCAGGTGGTGCAGTCTGGGGCTGAAGTA
AAGAAGCCTGGGTCCTCGGTGACGGTCTCCTGCAAGGCATCTGGA
GGCACCTTCAGCAACTATGCTATCAGCTGGGTGCGACAGGCCCCT
GGACAAGGGCTTGAGTGGATGGGAGGGATCATCCCTCTTTTTGGT
ACACCAACCTACTCACAGAACTTCCAGGGCAGAGTCACGATTACC
GCGGACAAATCCACCAGCACAGCCCACATGGAGCTGATCAGCCTG
AGATCTGAGGACACGGCCGTGTATTACTGTGCGACAGATCGCTAC
AGGCAGGCAAATTTTGACCGGGCCCGGGTTGGCTGGTTCGACCCC
TGGGGCCAGGGCACCCTGGTCACCGTCTCCTCAGCCTCCACCAAG
GGCCCATCGGTCTTCCCCCTGGCACCCTCCTCCAAGAGCACCTCT
GGGGGCACAGCGGCCCTGGGCTGCCTGGTCAAGGACTACTTCCCC
GAACCGGTGACGGTGTCGTGGAACTCAGGCGCCCTGACCAGCGGC
GTGCACACCTTCCCGGCTGTCCTACAGTCCTCAGGACTCTACTCC
CTCAGCAGCGTGGTGACCGTGCCCTCCAGCAGCTTGGGCACCCAG
ACCTACATCTGCAACGTGAATCACAAGCCCAGCAACACCAAGGTG
GACAAGAAAGTTGAGCCCAAATCTTGTGACAAAACTCACACATGC
CCACCGTGCCCAGCACCTGAACTCCTGGGGGGACCGTCAGTCTTC
CTCTTCCCCCCAAAACCCAAGGACACCCTCATGATCTCCCGGACC
CCTGAGGTCACATGCGTGGTGGTGGACGTGAGCCACGAAGACCCT
GAGGTCAAGTTCAACTGGTACGTGGACGGCGTGGAGGTGCATAAT
GCCAAGACAAAGCCGCGGGAGGAGCAGTACAACAGCACGTACCGT
GTGGTCAGCGTCCTCACCGTCCTGCACCAGGACTGGCTGAATGGC
AAGGAGTACAAGTGCAAGGTCTCCAACAAAGCCCTCCCAGCCCCC
ATCGAGAAAACCATCTCCAAAGCCAAAGGGCAGCCCCGAGAACCA
CAGGTGTACACCCTGCCCCCATCCCGGGATGAGCTGACCAAGAAC
CAGGTCAGCCTGACCTGCCTGGTCAAAGGCTTCTATCCCAGCGAC
ATCGCCGTGGAGTGGGAGAGCAATGGGCAGCCGGAGAACAACTAC
AAGACCACGCCTCCCGTGCTGGACTCCGACGGCTCCTTCTTCCTC
TACAGCAAGCTCACCGTGGACAAGAGCAGGTGGCAGCAGGGGAAC
GTCTTCTCATGCTCCGTGATGCATGAGGCTCTGCACAACCACTAC
ACGCAGAAGAGCCTCTCCCTGTCTCCGGGTAAATGA.

In embodiments, the IgG further comprises an immunoglobulin light chain (e.g., an immunoglobulin lambda chain) with at least about 85% amino acid sequence identity to GenBank Accession No. AAA02915.1, which is reproduced below. An exemplary immunoglobulin light chain polypeptide sequence is provided below.

(SEQ ID NO: 9)
MAWALLLLTLLTQDTGSWAQSALTQPASVSGSPGQSITISCTGTN
NDVGSYNLVSWYQQHPGKAPKIMIYEVSKRPSGVSNRFSGSKSGN
TASLTISGLQAEDEADYYCCSYAGSYTVVFGGGTKLTVLGQPKAA
PSVTLFPPSSEELQANKATLVCLISDFYPGAVTVAWKADSSPVKA
GVETTTPSKQSNNKYAASSYLSLTPEQWKSHRSYSCQVTHEGSTV
EKTVAPTECS

An exemplary polynucleotide encoding an immunoglobulin light chain is provided at GenBank Accession No. M87790.1, which is reproduced below.

(SEQ ID NO: 10)
ATGGCCTGGGCTCTGCTGCTCCTCACCCTCCTCACTCAGGACACA
GGGTCCTGGGCCCAGTCTGCCCTGACTCAGCCTGCCTCCGTGTCT
GGGTCTCCTGGACAGTCGATCACCATCTCCTGCACTGGAACCAAC
AATGATGTTGGGAGTTATAACCTTGTCTCCTGGTACCAGCAGCAC
CCAGGCAAAGCCCCCAAAATCATGATTTATGAGGTCAGTAAGCGG
CCCTCAGGGGTTTCTAATCGCTTCTCTGGCTCCAAGTCTGGCAAC
ACGGCCTCCCTGACAATCTCTGGGCTCCAGGCTGAGGACGAGGCT
GATTATTACTGCTGCTCATATGCAGGTAGTTACACTGTGGTTTTC
GGCGGAGGGACCAAACTGACCGTCCTAGGTCAGCCCAAGGCTGCC
CCCTCGGTCACTCTGTTCCCGCCCTCCTCTGAGGAGCTTCAAGCC
AACAAGGCCACACTGGTGTGTCTCATAAGTGACTTCTACCCGGGA
GCCGTGACAGTGGCCTGGAAGGCAGATAGCAGCCCCGTCAAGGCG
GGAGTGGAGACCACCACACCCTCCAAACAAAGCAACAACAAGTAC
GCGGCCAGCAGCTATCTGAGCCTGACGCCTGAGCAGTGGAAGTCC
CACAGAAGCTACAGCTGCCAGGTCACGCATGAAGGGAGCACCGTG
GAGAAGACAGTGGCCCCTACAGAATGTTCATAG.

By “Jagged-1 polypeptide” or “Jagged1 polypeptide” is meant an antibody or fragment thereof with at least about 85% amino acid sequence identity to UniProtKB/Swiss-Prot Accession No. P78504.3, which is reproduced below, and capable of activating notch receptors. An exemplary Jagged-1 polypeptide sequence is provided below (see UniProtKB/Swiss-Prot Accession No. P78504.3).

(SEQ ID NO: 11)
MRSPRTRGRSGRPLSLLLALLCALRAKVCGASGQFELEILSMQNV
NGELQNGNCCGGARNPGDRKCTRDECDTYFKVCLKEYQSRVTAGG
PCSFGSGSTPVIGGNTFNLKASRGNDRNRIVLPFSFAWPRSYTLL
VEAWDSSNDTVQPDSIIEKASHSGMINPSRQWQTLKQNTGVAHFE
YQIRVTCDDYYYGFGCNKFCRPRDDFFGHYACDQNGNKTCMEGWM
GPECNRAICRQGCSPKHGSCKLPGDCRCQYGWQGLYCDKCIPHPG
CVHGICNEPWQCLCETNWGGQLCDKDLNYCGTHQPCLNGGTCSNT
GPDKYQCSCPEGYSGPNCEIAEHACLSDPCHNRGSCKETSLGFEC
ECSPGWTGPTCSTNIDDCSPNNCSHGGTCQDLVNGFKCVCPPQWT
GKTCQLDANECEAKPCVNAKSCKNLIASYYCDCLPGWMGQNCDIN
INDCLGQCQNDASCRDLVNGYRCICPPGYAGDHCERDIDECASNP
CLNGGHCQNEINRFQCLCPTGFSGNLCQLDIDYCEPNPCQNGAQC
YNRASDYFCKCPEDYEGKNCSHLKDHCRTTPCEVIDSCTVAMASN
DTPEGVRYISSNVCGPHGKCKSQSGGKFTCDCNKGFTGTYCHENI
NDCESNPCRNGGTCIDGVNSYKCICSDGWEGAYCETNINDCSQNP
CHNGGTCRDLVNDFYCDCKNGWKGKTCHSRDSQCDEATCNNGGTC
YDEGDAFKCMCPGGWEGTTCNIARNSSCLPNPCHNGGTCVVNGES
FTCVCKEGWEGPICAQNTNDCSPHPCYNSGTCVDGDNWYRCECAP
GFAGPDCRININECQSSPCAFGATCVDEINGYRCVCPPGHSGAKC
QEVSGRPCITMGSVIPDGAKWDDDCNTCQCLNGRIACSKVWCGPR
PCLLHKGHSECPSGQSCIPILDDQCFVHPCTGVGECRSSSLQPVK
TKCTSDSYYQDNCANITFTFNKEMMSPGLTTEHICSELRNLNILK
NVSAEYSIYIACEPSPSANNEIHVAISAEDIRDDGNPIKEITDKI
IDLVSKRDGNSSLIAAVAEVRVQRRPLKNRTDFLVPLLSSVLTVA
WICCLVTAFYWCLRKRRKPGSHTHSASEDNTTNNVREQLNQIKNP
IEKHGANTVPIKDYENKNSKMSKIRTHNSEVEEDDMDKHQQKARF
AKQPAYTLVDREEKPPNGTPTKHPNWTNKQDNRDLESAQSLNRME
YIV

By “Jagged-1 polynucleotide” or “Jagged1 polynucleotide” is meant a nucleic acid molecule encoding a Jagged-1 polypeptide or fragment thereof.

By “Jagged1-Fc fusion polypeptide” is meant an antibody or fragment thereof with at least about 85% amino acid sequence identity to the below sequence, and capable of binding FGF2, HGF, and/or VEGF, and/or activating notch receptors. An exemplary Jagged1-FC fusion polypeptide sequence is provided below, which corresponds to residues S32-S1046 of a Jagged-1 polypeptide spliced to residues P100-K330 of an IgG Fc polypeptide via an Ile-Glu-Gly-Arg-Met-Asp (IEGRMD) linker (SEQ ID NO: 1).

(SEQ ID NO: 2)
SGQFELEILSMQNVNGELQNGNCCGGARNPGDRKCTRDECDTYFK
VCLKEYQSRVTAGGPCSFGSGSTPVIGGNTFNLKASRGNDRNRIV
LPFSFAWPRSYTLLVEAWDSSNDTVQPDSIIEKASHSGMINPSRQ
WQTLKQNTGVAHFEYQIRVTCDDYYYGFGCNKFCRPRDDFFGHYA
CDQNGNKTCMEGWMGPECNRAICRQGCSPKHGSCKLPGDCRCQYG
WQGLYCDKCIPHPGCVHGICNEPWQCLCETNWGGQLCDKDLNYCG
THQPCLNGGTCSNTGPDKYQCSCPEGYSGPNCEIAEHACLSDPCH
NRGSCKETSLGFECECSPGWTGPTCSTNIDDCSPNNCSHGGTCQD
LVNGFKCVCPPQWTGKTCQLDANECEAKPCVNAKSCKNLIASYYC
DCLPGWMGQNCDININDCLGQCQNDASCRDLVNGYRCICPPGYAG
DHCERDIDECASNPCLNGGHCQNEINRFQCLCPTGFSGNLCQLDI
DYCEPNPCQNGAQCYNRASDYFCKCPEDYEGKNCSHLKDHCRTTP
CEVIDSCTVAMASNDTPEGVRYISSNVCGPHGKCKSQSGGKFTCD
CNKGFTGTYCHENINDCESNPCRNGGTCIDGVNSYKCICSDGWEG
AYCETNINDCSQNPCHNGGTCRDLVNDFYCDCKNGWKGKTCHSRD
SQCDEATCNNGGTCYDEGDAFKCMCPGGWEGTTCNIARNSSCLPN
PCHNGGTCVVNGESFTCVCKEGWEGPICAQNTNDCSPHPCYNSGT
CVDGDNWYRCECAPGFAGPDCRININECQSSPCAFGATCVDEING
YRCVCPPGHSGAKCQEVSGRPCITMGSVIPDGAKWDDDCNTCQCL
NGRIACSKVWCGPRPCLLHKGHSECPSGQSCIPILDDQCFVHPCT
GVGECRSSSLQPVKTKCTSDSYYQDNCANITFTFNKEMMSPGLTT
EHICSELRNLNILKNVSAEYSIYIACEPSPSANNEIHVAISAEDI
RDDGNPIKEITDKIIDLVSKRDGNSIEGRMDPKSCDKTHTCPPCP
APELLGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALPAPIEKTISKAKGQPREPQVYTLPPSRDELTKNQVSL
TCLVKGFYPSDIAVEWESNGQPENNYKTTPPVLDSDGSFFLYSKL
TVDKSRWQQGNVFSCSVMHEALHNHYTQKSLSLSPGK

By “Jagged1-Fc fusion polynucleotide” is meant a nucleic acid molecule encoding a Jagged1-Fc fusion polypeptide or fragment thereof.

By “Hepatocyte Growth Factor (HGF)” is meant a protein or fragment thereof having at least about 85% identity to NCBI Accession No. NP_000592.3, reproduced below, that binds IgG. In embodiments, the HGF includes a histidine-tag (His-tag) comprising about or at least about 2, 3, 4, 5, 6, 7, 8, 9, or 10 contiguous histidine residues, optionally at the C-terminus or N-terminus of the IgG polypeptide sequence. An exemplary full-length human HGF polypeptide is provided below.

(SEQ ID NO: 12)
MWVTKLLPALLLQHVLLHLLLLPIAIPYAEGQRKRRNTIHEFKKS
AKTTLIKIDPALKIKTKKVNTADQCANRCTRNKGLPFTCKAFVFD
KARKQCLWFPFNSMSSGVKKEFGHEFDLYENKDYIRNCIIGKGRS
YKGTVSITKSGIKCQPWSSMIPHEHSFLPSSYRGKDLQENYCRNP
RGEEGGPWCFTSNPEVRYEVCDIPQCSEVECMTCNGESYRGLMDH
TESGKICQRWDHQTPHQURHKFLPERYPDKGFDDNYCRNPDGQPR
PWCYTLDPHTRWEYCAIKTCADNTMNDTDVPLETTECIQGQGEGY
RGTVNTIWNGIPCQRWDSQYPHEHDMTPENFKCKDLRENYCRNPD
GSESPWCFTTDPNIRVGYCSQIPNCDMSHGQDCYRGNGKNYMGNL
SQTRSGLTCSMWDKNMEDLHRHIFWEPDASKLNENYCRNPDDDAH
GPWCYTGNPLIPWDYCPISRCEGDTTPTIVNLDHPVISCAKTKQL
RVVNGIPTRTNIGWMVSLRYRNKHICGGSLIKESWVLTARQCFPS
RDLKDYEAWLGIHDVHGRGDEKCKQVLNVSQLVYGPEGSDLVLMK
LARPAVLDDFVSTIDLPNYGCTIPEKTSCSVYGWGYTGLINYDGL
LRVAHLYIMGNEKCSQHHRGKVTLNESEICAGAEKIGSGPCEGDY
GGPLVCEQHKMRMVLGVIVPGRGCAIPNRPGIFVRVAYYAKWIHK
IILTYKVPQS.

In one embodiment, an HGF fragment is a 29-35 kDa subunit (e.g., 31, 32, 33 kDa) or a 59-70 kDa subunit (e.g., 63, 64, 65, 66, 67 kDa subunit, Nakamura et al., 1989, Nature 342:440-443).

By “Hepatocyte Growth Factor (HGF) polynucleotide” is meant a nucleic acid molecule encoding an HGF polypeptide or fragment thereof. An exemplary human HGF polynucleotide is provided at NCBI Accession No. NM_000601, which is reproduced below.

(SEQ ID NO: 13)
GGGAGTTCAGACCTAGATCTTTCCAGTTAATCACACAACAAACTT
AGCTCATCGCAATAAAAAGCAGCTCAGAGCCGACTGGCTCTTTTA
GGCACTGACTCCGAACAGGATTCTTTCACCCAGGCATCTCCTCCA
GAGGGATCCGCCAGCCCGTCCAGCAGCACCATGTGGGTGACCAAA
CTCCTGCCAGCCCTGCTGCTGCAGCATGTCCTCCTGCATCTCCTC
CTGCTCCCCATCGCCATCCCCTATGCAGAGGGACAAAGGAAAAGA
AGAAATACAATTCATGAATTCAAAAAATCAGCAAAGACTACCCTA
ATCAAAATAGATCCAGCACTGAAGATAAAAACCAAAAAAGTGAAT
ACTGCAGACCAATGTGCTAATAGATGTACTAGGAATAAAGGACTT
CCATTCACTTGCAAGGCTTTTGTTTTTGATAAAGCAAGAAAACAA
TGCCTCTGGTTCCCCTTCAATAGCATGTCAAGTGGAGTGAAAAAA
GAATTTGGCCATGAATTTGACCTCTATGAAAACAAAGACTACATT
AGAAACTGCATCATTGGTAAAGGACGCAGCTACAAGGGAACAGTA
TCTATCACTAAGAGTGGCATCAAATGTCAGCCCTGGAGTTCCATG
ATACCACACGAACACAGCTTTTTGCCTTCGAGCTATCGGGGTAAA
GACCTACAGGAAAACTACTGTCGAAATCCTCGAGGGGAAGAAGGG
GGACCCTGGTGTTTCACAAGCAATCCAGAGGTACGCTACGAAGTC
TGTGACATTCCTCAGTGTTCAGAAGTTGAATGCATGACCTGCAAT
GGGGAGAGTTATCGAGGTCTCATGGATCATACAGAATCAGGCAAG
ATTTGTCAGCGCTGGGATCATCAGACACCACACCGGCACAAATTC
TTGCCTGAAAGATATCCCGACAAGGGCTTTGATGATAATTATTGC
CGCAATCCCGATGGCCAGCCGAGGCCATGGTGCTATACTCTTGAC
CCTCACACCCGCTGGGAGTACTGTGCAATTAAAACATGCGCTGAC
AATACTATGAATGACACTGATGTTCCTTTGGAAACAACTGAATGC
ATCCAAGGTCAAGGAGAAGGCTACAGGGGCACTGTCAATACCATT
TGGAATGGAATTCCATGTCAGCGTTGGGATTCTCAGTATCCTCAC
GAGCATGACATGACTCCTGAAAATTTCAAGTGCAAGGACCTACGA
GAAAATTACTGCCGAAATCCAGATGGGTCTGAATCACCCTGGTGT
TTTACCACTGATCCAAACATCCGAGTTGGCTACTGCTCCCAAATT
CCAAACTGTGATATGTCACATGGACAAGATTGTTATCGTGGGAAT
GGCAAAAATTATATGGGCAACTTATCCCAAACAAGATCTGGACTA
ACATGTTCAATGTGGGACAAGAACATGGAAGACTTACATCGTCAT
ATCTTCTGGGAACCAGATGCAAGTAAGCTGAATGAGAATTACTGC
CGAAATCCAGATGATGATGCTCATGGACCCTGGTGCTACACGGGA
AATCCACTCATTCCTTGGGATTATTGCCCTATTTCTCGTTGTGAA
GGTGATACCACACCTACAATAGTCAATTTAGACCATCCCGTAATA
TCTTGTGCCAAAACGAAACAATTGCGAGTTGTAAATGGGATTCCA
ACACGAACAAACATAGGATGGATGGTTAGTTTGAGATACAGAAAT
AAACATATCTGCGGAGGATCATTGATAAAGGAGAGTTGGGTTCTT
ACTGCACGACAGTGTTTCCCTTCTCGAGACTTGAAAGATTATGAA
GCTTGGCTTGGAATTCATGATGTCCACGGAAGAGGAGATGAGAAA
TGCAAACAGGTTCTCAATGTTTCCCAGCTGGTATATGGCCCTGAA
GGATCAGATCTGGTTTTAATGAAGCTTGCCAGGCCTGCTGTCCTG
GATGATTTTGTTAGTACGATTGATTTACCTAATTATGGATGCACA
ATTCCTGAAAAGACCAGTTGCAGTGTTTATGGCTGGGGCTACACT
GGATTGATCAACTATGATGGCCTATTACGAGTGGCACATCTCTAT
ATAATGGGAAATGAGAAATGCAGCCAGCATCATCGAGGGAAGGTG
ACTCTGAATGAGTCTGAAATATGTGCTGGGGCTGAAAAGATTGGA
TCAGGACCATGTGAGGGGGATTATGGTGGCCCACTTGTTTGTGAG
CAACATAAAATGAGAATGGTTCTTGGTGTCATTGTTCCTGGTCGT
GGATGTGCCATTCCAAATCGTCCTGGTATTTTTGTCCGAGTAGCA
TATTATGCAAAATGGATACACAAAATTATTTTAACATATAAGGTA
CCACAGTCATAGCTGAAGTAAGTGTGTCTGAAGCACCCACCAATA
CAACTGTCTTTTACATGAAGATTTCAGAGAATGTGGAATTTAAAA
TGTCACTTACAACAATCCTAAGACAACTACTGGAGAGTCATGTTT
GTTGAAATTCTCATTAATGTTTATGGGTGTTTTCTGTTGTTTTGT
TTGTCAGTGTTATTTTGTCAATGTTGAAGTGAATTAAGGTACATG
CAAGTGTAATAACATATCTCCTGAAGATACTTGAATGGATTAAAA
AAACACACAGGTATATTTGCTGGATGATAAAGATTTCATGGGAAA
AAAAATCAATTAATCTGTCTAAGCTGCTTTCTGATGTTGGTTTCT
TAATAATGAGTAAACCACAAATTAAATGTTATTTTAACCTCACCA
AAACAATTTATACCTTGTGTCCCTAAATTGTAGCCCTATATTAAA
TTATATTACATTTCAAAAAAAAAAAAAAAA.

By “vascular endothelial growth factor (VEGF) polypeptide” or “vascular endothelial growth factor (VEGFA) polypeptide” is meant a polypeptide or fragment thereof comprising an amino acid sequence with at least 85% amino acid sequence identity to NCBI Ref. Seq. Accession No. NP_001020537.2, which is reproduced below, and having angiogenic activity. An exemplary VEGF polypeptide is provided below.

(SEQ ID NO: 14)
MTDRQTDTAPSPSYHLLPGRRRTVDAAASRGQGPEPAPGGGVEGV
GARGVALKLFVQLLGCSRFGGAVVRAGEAEPSGAARSASSGREEP
QPEEGEEEEEKEEERGPQWRLGARKPGSWTGEAAVCADSAPAARA
PQALARASGRGGRVARRGAEESGPPHSPSRRGSASRAGPGRASET
MNFLLSWVHWSLALLLYLHHAKWSQAAPMAEGGGQNHHEVVKFMD
VYQRSYCHPIETLVDIFQEYPDEIEYIFKPSCVPLMRCGGCCNDE
GLECVPTEESNITMQIMRIKPHQGQHIGEMSFLQHNKCECRPKKD
RARQEKKSVRGKGKGQKRKRKKSRYKSWSVYVGARCCLMPWSLPG
PHPCGPCSERRKHLFVQDPQTCKCSCKNTDSRCKARQLELNERTC
RCDKPRR.

By “vascular endothelial growth factor (VEGF) polynucleotide” or “vascular endothelial growth factor (VEGFA) polynucleotide” is meant a polynucleotide or fragment thereof encoding VEGF. An exemplary VEGF polynucleotide is provided at NCBI Ref. Seq. Accession No. NM_001025366.3, which is reproduced below. An exemplary VEGF polynucleotide is provided below.

(SEQ ID NO: 15)
CTGACGGACAGACAGACAGACACCGCCCCCAGCCCCAGCTACCAC
CTCCTCCCCGGCCGGCGGCGGACAGTGGACGCGGCGGCGAGCCGC
GGGCAGGGGCCGGAGCCCGCGCCCGGAGGCGGGGTGGAGGGGGTC
GGGGCTCGCGGCGTCGCACTGAAACTTTTCGTCCAACTTCTGGGC
TGTTCTCGCTTCGGAGGAGCCGTGGTCCGCGCGGGGGAAGCCGAG
CCGAGCGGAGCCGCGAGAAGTGCTAGCTCGGGCCGGGAGGAGCCG
CAGCCGGAGGAGGGGGAGGAGGAAGAAGAGAAGGAAGAGGAGAGG
GGGCCGCAGTGGCGACTCGGCGCTCGGAAGCCGGGCTCATGGACG
GGTGAGGCGGCGGTGTGCGCAGACAGTGCTCCAGCCGCGCGCGCT
CCCCAGGCCCTGGCCCGGGCCTCGGGCCGGGGAGGAAGAGTAGCT
CGCCGAGGCGCCGAGGAGAGCGGGCCGCCCCACAGCCCGAGCCGG
AGAGGGAGCGCGAGCCGCGCCGGCCCCGGTCGGGCCTCCGAAACC
ATGAACTTTCTGCTGTCTTGGGTGCATTGGAGCCTTGCCTTGCTG
CTCTACCTCCACCATGCCAAGTGGTCCCAGGCTGCACCCATGGCA
GAAGGAGGAGGGCAGAATCATCACGAAGTGGTGAAGTTCATGGAT
GTCTATCAGCGCAGCTACTGCCATCCAATCGAGACCCTGGTGGAC
ATCTTCCAGGAGTACCCTGATGAGATCGAGTACATCTTCAAGCCA
TCCTGTGTGCCCCTGATGCGATGCGGGGGCTGCTGCAATGACGAG
GGCCTGGAGTGTGTGCCCACTGAGGAGTCCAACATCACCATGCAG
ATTATGCGGATCAAACCTCACCAAGGCCAGCACATAGGAGAGATG
AGCTTCCTACAGCACAACAAATGTGAATGCAGACCAAAGAAAGAT
AGAGCAAGACAAGAAAAAAAATCAGTTCGAGGAAAGGGAAAGGGG
CAAAAACGAAAGCGCAAGAAATCCCGGTATAAGTCCTGGAGCGTG
TACGTTGGTGCCCGCTGCTGTCTAATGCCCTGGAGCCTCCCTGGC
CCCCATCCCTGTGGGCCTTGCTCAGAGCGGAGAAAGCATTTGTTT
GTACAAGATCCGCAGACGTGTAAATGTTCCTGCAAAAACACAGAC
TCGCGTTGCAAGGCGAGGCAGCTTGAGTTAAACGAACGTACTTGC
AGATGTGACAAGCCGAGGCGGTGA.

By “agent” is meant any small molecule chemical compound, antibody, nucleic acid molecule, polypeptide, polypeptide complex, or fragments thereof. Non-limiting examples of agents include polypeptide complexes, such as those containing FGF2 and IgG, or fragments thereof, optionally where the complexes further contain VEGF, HGF, or fragments thereof.

By “ameliorate” is meant decrease, suppress, attenuate, diminish, arrest, or stabilize the development or progression of a disease. In embodiments, a composition of the invention ameliorates a condition associated with reperfusion injury, hypofusion, ischemic injury, and/or low/no-reflow. In some embodiments, the composition of the invention ameliorates a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), symptoms thereof, and the like. In embodiments, the hypofusion is cerebral hypofusion, tissue hypofusion, and/or organ hypofusion.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. In embodiments, the compositions and methods employ a basic fibroblast growth factor (FGF2), hepatocyte growth factor (HGF), vascular endothelial growth factor (VEGF), and/or IgG analog (e.g., various glycosylated forms including, as a non-limiting example, sialylated forms).

In this disclosure, “comprises,” “comprising,” “containing,” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected.

By “disease” is meant any condition or disorder that damages or interferes with the normal function of a cell, tissue, or organ. Examples of diseases include any disease or injury that results in a reduction in cell number or biological function, including a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like. The disease in embodiments is associated with myocardial infarction (MI), reperfusion injury, hypofusion, ischemic injury, and/or low/no-reflow. Diseases include any ischemic event that causes tissue damage. In embodiments, the hypofusion is cerebral hypofusion, tissue hypofusion, and/or organ hypofusion.

By “effective amount” is meant the amount of an agent required to ameliorate the symptoms of a disease relative to an untreated patient. In one embodiment, the compositions of the invention comprise an effective amount of an isolated and/or purified complex containing FGF2 and IgG, or fragments thereof, optionally where the complex further contains VEGF, HGF, or fragments thereof.

In embodiments, the complexes are used for the therapeutic treatment of ischemic injury. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In embodiments, an effective amount of a composition of the invention contains about or at least about 0.0001, 0.0005, 0.001, 0.0025, 0.005, 0.0075, 0.01, 0.02, 0.03, 0.04, 0.05, 0.06, 0.07, 0.08, 0.09, 0.10, 0.2, 0.3, 0.4, or 0.5 mg of FGF2, HGF, VEGF, and/or IgG per kg of a subject to which the composition is administered. In embodiments, the subject weights 30 kg, 40 kg, 50 kg, 60 kg, 70 kg, 80 kg, 90 kg, 100 kg, 110 kg, 120 kg, 130 kg, 140 kg, 150 kg, 160 kg, 170 kg, 180 kg, 190 kg, or 200 kg.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In one embodiment, an HGF fragment is a 29-35 kDa subunit (e.g., 31, 32, 33 kDa) or a 59-70 kDa subunit (e.g., 63, 64, 65, 66, 67 kDa subunit) as measured by SDS PAGE.

By “hypofusion” is meant a reduced amount of blood flow. In embodiments, hypofusion is distinct from frank ischemia or complete block of flow. In embodiments, the reduced amount of blood flow is relative to a reference. In some instances, ischemia is associated with hypofusion. Non-limiting examples of hypofusion include cerebral hypofusion, tissue hypofusion, and/or organ hypofusion.

By “increase” is meant to alter positively by at least 5%. An increase may be by 5%, 10%, 25%, 30%, 50%, 75%, or even by 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis. When a cellular factor is “isolated” from a cultured epicardial progenitor cell the cellular factor is typically separated from cells and cellular debris. It need not be purified to homogeneity. In fact, the composition comprising an isolated cellular factor typically comprises any number of cellular factors whose presence contributes to the biological activity (e.g., growth promoting, survival promoting, or proliferation promoting activity) of the composition. In one embodiment, a composition of the invention comprises or consists of conditioned media from which cells and cellular debris have been removed.

By “marker” is meant any protein or polynucleotide having an alteration in expression level or activity that is associated with a developmental state, condition, disease, or disorder. A non-limiting example of a marker of plasma injury (e.g., vascular injury after ST segment elevation myocardial infarction (STEMI)) is Angiopoietin-2 (Angpt-2).

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant a negative alteration of at least 10%, 25%, 50%, 75%, or 100%. In particular, vascular permeability may be reduced relative to a reference (e.g., untreated control vessel) by at least about 5%, 10%, 25%, 30%, 50%, 75%, or even by 100%.

By “reference” is meant a standard or control condition. Non-limiting examples of a reference include a subject not having a disease, a healthy cell or subject, or a cell or subject not exposed to a particular stress (e.g., low oxygen stress, low/no-reflow, reperfusion, hypofusion, ischemic injury, and/or myocardial infarction (MI)), or an untreated corresponding control subject (e.g., having an untreated MI, hypofusion, ischemic injury, or reperfusion injury).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “repair” is meant to ameliorate damage or disease in a tissue or organ.

By “tissue” is meant a collection of cells having a similar morphology and function.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

By “vascular integrity” is meant maintenance of flow in a vessel without leakage.

Exemplary vessels include arteries, arterioles, veins, venules, capillaries, and microvessels. The flow in embodiments is fluid flow or flow of cells. In embodiments, the leakage is leakage of a fluid and/or cells.

By “vascular permeability” is meant leakage from a vessel into the surrounding environment. The leakage in embodiments is of blood or another bodily fluid.

By “subject” is meant an animal. The animal can be a mammal. The mammal can be a human or non-human mammal, such as a bovine, equine, canine, ovine, rodent, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1E provide images and bar plots showing intracoronary administration of human HGF/IgG complexes from the PCI guide catheter in a pre-clinical porcine model of MI with reperfusion. FIG. 1A, left and middle panels: Representative images from fluoroscopic angiography showing guide catheter with contrast dye. FIG. 1A, right panel: Image showing intracoronary infusion of HGF/IgG complexes or vehicle from the indwelling guide catheter at time of reperfusion (60 min post-ischemia). Pigs were treated with 62.5 μg HGF and 105 μg mixed polyclonal IgG, representing a 1:1:molar ratio. FIGS. 1B and 1C, left panels: Images taken at 24 hours after MI, reperfusion, and treatment, Evan's blue dye stain showed areas with adequate perfusion (generally, darker shading in lower portion of the images) and low perfusion (generally, darker shading in the upper portion of the images and lighter shading; a.k.a. Area At Risk, AAR) in representative vehicle-treated heart section (FIG. 1B) and HGF/IgG-treated heart section (FIG. 1C). FIGS. 1B and 1C, right panels: Images where TTC stain showed living muscle tissue (darkest shading) and necrotic, dying tissue (lighter shading; a.k.a. Ischemic Area, IA). FIG. 1D: Bar graphs showing quantification of myocardial salvage following MI/R and treatment with vehicle or 3 different doses of HGF/IgG complexes (Dose A, 31.25 μg HGF/52.5 μg IgG; Dose B, 62.5 μg HGF/105 μg IgG; Dose C, 125 μg HGF μg/210 μg IgG. One-way ANOVA with multiple comparisons. ***P<0.001. Myocardial salvage=AAR-IA/AAR. FIG. 1E: Bar graphs showing reduction in area of myocardial tissue with infarction, 24 hrs after MI/R and treatment with Dose B. Data are mean±SD. n=4-11 pigs per group. 2-tailed, unpaired T test. *P=0.05.

FIGS. 2A and 2B provide images showing vascular and myocardial distribution of HGF/IgG complexes 24 hrs after intracoronary infusion. Note light grey spots (ALEXA 594-conjugated secondary antibody) on endothelial wall and throughout myocardium in (FIG. 2B) that were not present in vehicle-treated heart (FIG. 2A). Prior to staining with secondary antisera and DAPI, both A and B were stained with polyclonal primary antisera against 6×His-tag (SEQ ID NO: 16) to detect recombinant human HGF. Hearts in FIGS. 2A and 2B are representative of n=3 pigs per group that were stained.

FIG. 3 provides a plot showing multiple administrations of HGF/IgG complexes do not induce HGF-specific auto-antibodies in rats subjected to myocardial ischemia-reperfusion. After MI/R surgery or sham surgery, blood plasma was obtained from rats weekly and assessed for circulating antibodies specific to rat HGF. Animals received 3 separate treatments: one at reperfusion, one on day 3, and one on day 5 post-MI/R with either vehicle (containing IgG), or HGF/IgG. HGF/IgG-treated animals showed no auto-immune reactivity to HGF. However, weekly addition of human FGF2 to HGF/IgG complexes (positive xenogeneic control) induced antibodies to rat HGF, as shown by a mild immune response at weeks 3 and 4. Data represent mean±SD and were analyzed by repeated measures two-way ANOVA. n=5 animals, **P<0.01.

FIGS. 4A-4C provide a gel image and bar graphs showing FGF2/IgG complexes protect primary human cardiac microvascular endothelial cells against simulated ischemia for 48-72 hrs. FIG. 4A: Image of a native isoelectric focusing gel to detect protein complexes. Lanes: 1) free IgG (alone); 2) free FGF2 (alone); 3) FGF2/IgG complexes. Gel was stained for 30 min with Coomassie brilliant blue and de-stained overnight in 10% acetic acid. Boxed area: note that the protein band expected for free IgG is absent in the right-hand lane with FGF2/IgG complexes. FIG. 4B: Bar graphs showing that MTS assay demonstrated increased cell viability conferred by FGF2/IgG and HGF/IgG complexes relative to IgG alone (matched dose) after 48 hours of exposure to 1% oxygen and nutrient deprivation. Data are mean±S.D. n=4 experimental replicates. *P<0.05, compared with IgG. FIG. 4C: Bar graph demonstrating CyQuant assays showed that FGF2/IgG and HGF/IgG complexes protected human cardiac microvascular endothelial cells against simulated ischemia for 72 hours. Data are mean±S.D and were analyzed by one-way ANOVA with post-hoc comparisons. n=6 experimental replicates.**P<0.01, compared with IgG.

FIGS. 5A-5D provide domain structure schematics and images from three-dimensional modeling of proposed growth factor:IgG complexes. FIG. 5A, top: Schematic representations of full-length FGF2 and FGFR1. FIG. 5A, bottom: Ribbon representations of FGF2 (residues 158-286, light grey) bound to FGFR1 (dark grey) and heparin (sticks)(published crystal structure, PDB ID 1FQ9). Note the binding of FGF2 proteins to FGFR1 occurs within immunoglobulin-like sub-domains. FIG. 5B, top: Schematic representations of the proposed binding of FGF2 to the IgG1 Fc domain. FIG. 5B, bottom: Ribbon model of proposed binding of FGF2 (light grey) to the Fc domain of IgG1 (dark grey) within FGF2/IgG complexes. Note that based on similarities to FGF2:FGFR1 binding, it is possible that the Fc domain of IgG may accommodate the binding of two FGF2 ligands. FIG. 5C, top: Schematic representations of the HGF pan domain and IgG1. FIG. 5C, bottom: Ribbon model of proposed binding of HGF pan domain (lighter grey, upper left and lower right of the structure) to the Fc domain of IgG1 (darker grey) in HGF/IgG complexes. Note that it is also possible that the Fc domain of IgG may accommodate the binding of two HGF ligands. FIGS. 5B and 5C: hexagon (top) and sticks (bottom)=glycosylation. FIG. 5D: Electrostatic surface potential models of FGF2 and HGF pan domain bound to heparin (sticks)(PDB IDs 1FQ9 and 1GMO, respectively). Note growth factors have heparin binding domains that are thought to bind within immunoglobulin-like domains of their respective RTK receptors. Not intending to be bound by theory, basic patches (dark grey surface) may be involved in the binding of FGF2 and HGF in the described complexes. PK=protein kinase, HC=heavy chain, K1-K4=Kringle domains, SPH=serine protease homology domain, alpha=HGF alpha chain, beta=HGF beta chain. Domains not shown in ribbon diagrams for FIG. 5A are the first Ig domain of FGFR1 and the PK domain of FGFR1, for FIG. 5B are the first two Ig domains of IgG1, and for FIG. 5C are all domains following the Pan domain of HGF and the first two Ig domains of IgG1.

FIGS. 6A-6E provide a gel image, bar graphs, and ribbon representations of protein structures showing generation of recombinant human FGF2/Jagged1-Fc complexes. FIG. 6A: Native isoelectric focusing gel to detect protein complexes. Lanes: 1) free Jagged1-Fc fusion protein (alone); 2) FGF2/Jagged1-Fc complex. Boxed area: note that the protein band expected for free Jagged1-Fc IgG is absent in the right-hand lane with FGF2/Jagged1-Fc complexes. Thus, complexation with FGF2 retarded the migration of Jagged1-Fc. FIG. 6B: Bar graph showing ELISA data for FGF2 to assay complex formation. FGF2 was incubated either alone or with Jagged1-Fc-His, followed by addition of anti-His-biotin. Streptavidin-agarose was used to pulldown complexes and 0.5% Sodium deoxycholate was used to dissociate protein-protein interactions prior to FGF2 ELISA. Data in (FIGS. 6B and 6C) are mean±S.D. and were analyzed by one-way ANOVA with post-hoc comparisons. n=2-3 experimental replicates. **P<0.01. FIG. 6C: Bar graph showing addition of excess heparin (50 μg) during initial incubation of FGF2 with Jagged-Fc-His prevents complex formation. **P<0.01. FIG. 6D: Ribbon representation of modeled Jagged1-Fc fusion protein homodimer. FIG. 6E: Ribbon representation of modeled Jagged1-Fc fusion in complex with FGF2.

### DETAILED DESCRIPTION OF THE INVENTION

The invention features, among other things, isolated protein complexes and compositions and methods for use thereof. In embodiments, the isolated protein complexes are used for treating conditions associated with reperfusion injury, hypofusion, and/or low/no-reflow. The isolated protein complexes comprise a fusion protein complexed with basic fibroblast growth factor (FGF2), hepatocyte growth factor (HGF), or vascular endothelial growth factor (VEGF). The fusion protein contains an immunoglobulin G (IgG) Fc domain fused to a polypeptide (e.g., Jagged1, a growth factor, or a cytokine). In embodiments, reperfusion injury, hypofusion, ischemic injury, and/or low/no-reflow is associated with a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like. In embodiments, the hypofusion is cerebral hypofusion, tissue hypofusion, and/or organ hypofusion.

The present invention is based, at least in part, upon the discovery that fusion proteins containing the Fc domain from IgG1 can form complexes with FGF2. In particular, in the Examples provided herein, it is shown that FGF2 formed complexes with Jagged1-Fc fusion polypeptides.

In instances, the compositions are vaso- and/or cardioprotective and/or associated with an increase in vascular integrity and/or preservation of tissue (e.g., cardiac tissue) jeopardized by reperfusion injury, hypofusion, ischemic injury, and/or low/no re-flow. Compositions of the present invention reduce infarct size and improve patient outcomes after myocardial infarction. Compositions of the invention provide significant myocardial salvage and/or reduce infarct size. In embodiments, reperfusion injury, hypofusion, ischemic injury, and/or low/no-reflow is associated with a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like. In embodiments, the hypofusion is cerebral hypofusion, tissue hypofusion, and/or organ hypofusion.

The combination of FGF2 and HGF is angiogenic (i.e., promotes blood vessel sprouting from pre-existing vessels). Moreover, in embodiments, the combination of FGF2 and HGF is associated with generation of stable vessels that last longer than those formed with HGF alone. The complexes of the present invention (e.g., complexes containing FGF2 and a fusion protein comprising an Fc domain (e.g., an Fc domain joined to a Jagged1, a growth factor, or a cytokine polypeptide or fragments thereof), or fragments thereof, optionally where the complexes further contain VEGF, HGF, or fragments thereof) can be easily produced in a clinical setting from FGF2, HGF, and the fusion protein. In some instances, the fusion protein contains a Jagged1, a growth factor, and/or a cytokine domain. The complex containing FGF2 and the fusion protein containing an Fc domain forms a basic biochemical infrastructure from which to build custom agonist or antagonist signaling complexes with ligands of desired properties, for particular indications (e.g., complexes containing FGF2 and the fusion protein, or fragments thereof, optionally where the complexes further contain VEGF, HGF, or fragments thereof). This biochemical infrastructure is likely to allow for the preparation of custom complexes constituting designer biologic drugs that target particular biological processes to obtain a desired outcome.

Accordingly, the invention provides complexes comprising FGF2, HGF, and a fusion protein containing an Fc domain (e.g., an IgG1 Fc domain), and methods of using such complexes to treat a variety of indications associated with hypofusion and/or ischemic injury (e.g., reperfusion injury, hypofusion, and/or low/no-reflow associated with a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like). In embodiments, the hypofusion is cerebral hypofusion, tissue hypofusion, and/or organ hypofusion.

### Reperfusion Injury and Low/No-Reflow

Revascularization of the epicardial coronaries is important for patient survival after MI. Paradoxically, however, myocardial reperfusion also causes injury, killing vascular endothelial cells, cardiomyocytes and other vulnerable cells, which greatly reduces the benefits of reperfusion therapy (Yellon & Hausenloy, 2007). Lethal reperfusion injury, or the injury and eventual death of cardiomyocytes that were viable immediately prior to reperfusion, contributes up to 50% of final infarct size (Garcia-Dorado, Ruiz-Meana, & Piper, 2009). In addition to PCI and thrombolysis to treat MI, reperfusion injury also occurs during coronary artery bypass (CABG) procedures and heart transplantation (Verma et al., 2002).

Reperfusion injury and low/no-reflow after myocardial infarction and percutaneous coronary intervention (PCI) increase infarct size and mortality. Not being limited by theory, microvascular integrity and extent of no/low-reflow are determinants of infarct expansion. Consequently, numerous mechanical (e.g. thrombectomy) and pharmacological approaches (e.g. adenosine, nitroprusside, nicorandil, verapamil) have been tested to prevent or alleviate no/low-reflow. The 5 year prognosis for mortality in no-reflow patients with acute ST segment elevation myocardial infarction (STEMI) remains poor relative to those with reflow, and all phase III clinical trials have failed to provide long-term benefit. Among a group of 1,406 patients with ST segment elevation myocardial infarction (STEMI) that underwent percutaneous coronary intervention (PCI), 410 (29%) were diagnosed with no-reflow (Ndrepepa et al., J. Am. Coll. Cardiol. 55, 2383-2389 (2010)). Kaplan-Meier estimates of 5-year mortality were 18.2% for patients with no-reflow and 9.5% for reflow patients. Infarct size was highly correlated to incidence of no-reflow. The mean infarct size in no-reflow patients was 15.0% of the left ventricle whereas in reflow patients it was 8% of the left ventricle. In addition to increased infarct size, patients with no/low-reflow have a higher incidence of early post-infarction complications (e.g. arrhythmias, pericardial effusion, early congestive heart failure), and adverse left ventricular remodeling compared with those with reflow.

Angiopoietin-2 (Angpt-2) is a potential diagnostic plasma marker for vascular injury after ST segment elevation myocardial infarction (STEMI) (Tarikuz Zaman A K M, French C J, Spees J L, Binbrek A S, Sobel B E, “Vascular rhexis in mice subjected to non-sustained myocardial ischemia and its therapeutic implications” Exp. Biol. Med. (Maywood) 236:598-603 (2011)). Not being bound by theory, Angpt-2 is released into the circulation in a bi-phasic pattern after myocardial infarction (MI); first from necrotic endothelial cells early after ischemia/reperfusion and later during angiogenesis for tissue repair. Creatine kinase is an enzyme released from necrotic cardiac myocytes after MI. Notably, at 48 hr after MI, plasma Angpt-2 levels in patients were are correlated to infarct size determined by analysis of circulating creatine kinase activity (MB isoform)(P=0.0017) (Tarikuz Zaman A K M, French C J, Spees J L, Binbrek A S, Sobel B E, “Vascular rhexis in mice subjected to non-sustained myocardial ischemia and its therapeutic implications” Exp. Biol. Med. (Maywood) 236:598-603 (2011)). Circulating Angpt-2 levels correlate to peak levels of cardiac troponin T, a marker widely used to estimate infarct size. Thus, endothelial cell/vascular injury is a predictor of infarct expansion/size and a potential therapeutic target.

Complexes containing FGF2, HGF, and a fusion protein containing an Fc domain (e.g., an Fc domain joined to a Jagged1, a growth factor, or a cytokine polypeptide or fragments thereof) can reduce reperfusion injury after myocardial infarction and percutaneous coronary intervention (PCI).

### Cardioprotective Treatments and PCI

Primary percutaneous coronary intervention (PCI) is the Standard of Care (SoC) for ST segment elevation myocardial infarction (STEMI). Typically, the percutaneous coronary intervention (PCI) guide catheter is removed immediately after stenting. However, this means that a valuable opportunity is missed to directly treat the affected arteries, arterioles, and capillaries downstream of the occlusive site. Because up to 50% of final infarct size in patients is determined by reperfusion injury and the degree of low/no-reflow after myocardial infarction (MI) and percutaneous coronary intervention (PCI), treatment strategies that are vaso-protective and/or angiogenic (e.g., complexes containing FGF2 and a fusion protein containing an Fc domain (e.g., an Fc domain joined to a Jagged1, a growth factor, or a cytokine polypeptide or fragments thereof), or fragments thereof, optionally where the complexes further contain VEGF, HGF, or fragments thereof) have great potential to reduce or prevent infarct expansion, decrease final infarct size, and improve patient outcomes. In embodiments, a complex containing FGF2 and a fusion protein containing an Fc domain, or fragments thereof, optionally where the complexes further contain VEGF, HGF, or fragments thereof, is administered to a subject as biologic drug treatments integrated into the PCI procedure. Because ischemic tissue injury increases over time, a major concern with cardioprotective treatments is whether or not they increase time to stenting and reperfusion. Importantly, compositions of the present invention (e.g., compositions containing complexes containing FGF2 and a fusion protein containing an Fc domain, or fragments thereof, optionally where the complexes further contain VEGF, HGF, or fragments thereof) can be delivered directly into the cardiac circulation through the indwelling PCI guide catheter—after/during stenting and/or before/after reperfusion. In embodiments, the compositions of the present disclosure are safe, well tolerated, and require only a limited time (e.g., 1, 2, 3, 4, 5, 6, 7, 8, 9, 10, 15, or 20 minutes) to infuse prior to removal of the guide catheter.

While about 30 to about 50% of patients exhibit low/no-reflow after myocardial infarction (MI), all patients undergoing percutaneous coronary intervention (PCI) have reperfusion injury that could be treated. Combining administration of compositions of the present invention (e.g., compositions containing complexes containing FGF2 and a fusion protein containing an Fc domain, or fragments thereof, optionally where the complexes further contain VEGF, HGF, or fragments thereof) with primary PCI as standard of care could benefit over 800,000 Americans each year and millions of myocardial infarction (MI) patients worldwide. Given the rapidly growing global market for cardiovascular disease, agents of the present disclosure are likely to be game changing, blockbuster drugs.

Further, growth factor-based treatments for ischemia-reperfusion and tissue injury have been studied in numerous animal models and proof-of-concept clinical trials that demonstrated positive safety profiles. But most growth factors administered intra-arterially or IV exhibited unfavorable pharmacokinetics (e.g. short half-lives and rapid clearance) and limited efficacy (Appasamy et al., 1993; Lazarous et al., 1997; Simons et al., 2002; Yang et al., 2009). Treatment with factors such as FGF2 or HGF is highly attractive due to their potential for synergistic signaling through common protective and pro-survival pathways such as Ras/MAPK and/or PI3K/Akt (Banquet et al., 2011; Bard et al., 2020; Koraishy et al., 2014; Liao et al., 2007b). The engagement of these pathways in myocardium with infarction has potential to protect vulnerable cardiomyocytes and endothelial cells from death, reduce vascular leak, and encourage angiogenesis for repair and regeneration.

### Fusion Proteins

The invention provides fusion proteins containing an Fc domain (e.g., an IgG1 Fc domain), or fragments thereof, is fused to a polypeptide, or fragments thereof. The Fc domain may be joined to a C-terminus or an N-terminus of the polypeptide. In some instances, the polypeptide is Jagged1, a growth factor, a cytokine polypeptide, or fragments thereof. In embodiments, the fusion protein is a Jagged1-Fc polypeptide. Jagged1 is a Notch1 ligand that controls stem cell self-renewal and tissue regeneration.

In some cases, the Fc domain is joined to the polypeptide by a linker. In embodiments, the linker contains about, at least about, and/or no more than about 1, 2, 3, 4, 5, 6, 7, 8, 9, 10, 15, 20, 25, 30, 35, 40, 45, or 50 amino acids. A non-limiting example of a linker amino acid sequence is IEGRMD (SEQ ID NO: 1). Exemplary linkers and methods for the selection and design of linkers include those described in Chen, et al. “Fusion Protein Linkers: Property, Design and Functionality,” Adv Drug Deliv Rev. 65:1357-1369 (2013).

The fusion proteins are capable of forming a complex with FGF2, HGF, and/or VEGF.

### Complexes

The present invention provides agents comprising complexes containing a fusion protein containing an Fc domain (e.g., an Fc domain joined to a Jagged1, a growth factor, or a cytokine polypeptide or fragments thereof), and basic fibroblast growth factor (FGF2), hepatocyte growth factor (HGF), and/or vascular endothelial growth factor (VEGF) (e.g., complexes containing FGF2 and the fusion protein, or fragments thereof, optionally where the complexes further contain VEGF, HGF, or fragments thereof). In embodiments, the complexes are self-assembling (i.e., they form spontaneously in solution after addition of FGF2). Not being bound by theory, FGF2 seeds complex formation. In embodiments, the complexes are formed in a method that does not involve a concentrating and/or centrifugation step. Various complexes of the present invention are formed without the need for any concentration and/or centrifugation step, although preformed complexes can optionally be concentrated using various methods (e.g., ultracentrifugation).

Not being bound by theory, the heparin-mimetic binding activity in the Immunoglobulin G (IgG) Fc domain forms therapeutic protein complexes with angiogenic, heparin-binding growth factors such as hepatocyte growth factor (HGF) and basic Fibroblast Growth Factor (FGF2). Mammalian IgG molecules possess N-glycosylation sites in the Fc domain that affect their function(s). The sugar molecules located at these sites can be further modified by fucosylation, galactosylation, and sialylation. Not wishing to be bound by theory, heparin molecules are negatively-charged polysaccharides that promote the formation of anti-thrombin:thrombin protein complexes. These complexes deactivate thrombin and prevent blood coagulation. Due to over-expansion of B cell clones that express particular IgG glycoforms, many cancer patients with multiple myeloma present with bleeding complications, in part, due to formation of IgG1:anti-thrombin complexes that inhibit thrombin, thereby increasing time to clot (similar in effect to addition of heparin). Not being bound by theory, the agents of the present invention may perform by a similar mechanism, i.e. the glycosylated, negatively-charged Fc domain of IgG may attract the heparin-binding domains of HGF and FGF2.

In embodiments, purified recombinant FGF2, HGF, and/or VEGF is combined with a fusion protein containing an Fc domain isolated and/or purified from human sources or other mammalian sources (i.e. rat, mouse, rabbit, pig, goat). In this manner, complexes (e.g., complexes containing FGF2 and a fusion protein containing an Fc domain, or fragments thereof, optionally where the complexes further contain VEGF, HGF, or fragments thereof) may be assembled by various means. Non-limiting examples of such means include spontaneous formation by contacting two polypeptides with one another (i.e., with no concentration step), with concentration by filtration, with centrifugation, column chromatography, changes in temperature or density, or by effectively increasing concentration through addition of particular molecules such as dextran sulphate or polyethylene glycol as is standard in the art in methods associated with developing probes for in situ hybridization. Alternatively, more simple means of forming complexes may be employed such as through altering the effective concentrations of polypeptides forming the complex (e.g., FGF2, HGF, VEGF, and/or a fusion protein containing an Fc domain).

In embodiments, the complexes (e.g., complexes containing FGF2 and a fusion protein containing an Fc domain, or fragments thereof, optionally where the complexes further contain VEGF, HGF, or fragments thereof) provide enhanced vaso-protection compared with free (i.e., non-complexed) fibroblast growth factor (FGF2), or free hepatocyte growth factor (HGF), alone or in combination. The methods and compositions of the present invention preserve vascular integrity and improve cardiac function. Not being bound by theory, the complexes are associated with a reduction in endothelial cell injury and vascular permeability. In some instances, the complexes are associated with significant myocardial salvage and/or reduced infarct size. In embodiments, the complexes are associated with an increase in endothelial cell survival. In some instances, the complexes are associated with activation (e.g., via phosphorylation) of c-Met (an HGF receptor), Ryk (a Wingless (Wnt) co-receptor; also known as “related to tyrosine kinase”), and/or FGFR. Complex formation increases the half-life of FGF2, VEGF, and/or HGF relative to free FGF2, free VEGF, or free HGF.

Since the complexes containing FGF2 and the fusion proteins form spontaneously in buffered saline solution without the need for centrifugation, fresh complexes (e.g., complexes containing FGF2 and a fusion protein containing an Fc domain, or fragments thereof, optionally where the complexes further contain VEGF, HGF, or fragments thereof) could be generated in the clinical setting without the need for specialized equipment and by comparatively simple methods (e.g., methods involving less steps). In addition, while HGF alone induces angiogenesis, the combination of HGF and FGF2 generates more durable capillaries and microvasculature. Thus, whereas complexes containing HGF and a fusion protein containing an Fc domain and complexes containing FGF2, HGF, and a fusion protein containing an Fc domain combined each provide vaso-protection, cardio-protection and angiogenesis, the complexes containing FGF2, HGF, and a fusion protein containing an Fc domain provide additional benefit(s) for patients in terms of durable blood vessel growth, myocardial perfusion, and cardiac function.

The complexes of the invention comprise a fusion protein containing an Fc domain in complex with an additional polypeptide(s) (e.g., FGF2, HGF, and/or VEGF). In embodiments, a composition of the invention comprise a fusion protein containing an Fc domain in an amount such that the molar ratio of the fusion protein to the additional polypeptide(s) in the composition is about or at least about 0.5, 1, 1.5, 2, 2.5, 3, 3.5, 4, or 5. In some instances, the compositions comprise equimolar amounts of the additional polypeptides.

In embodiments, complexes containing FGF2, HGF, and a fusion protein containing an Fc domain are at least as vaso- and cardioprotective as complexes containing HGF and IgG.

### Polypeptide Production

In general, polypeptides of the invention may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins (e.g., an Fc domain joined to a Jagged1, a growth factor, or a cytokine polypeptide or fragments thereof) with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once a recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs.

### Therapeutic Methods

Compositions comprising complexes comprising FGF2, a fusion protein containing an Fc domain (e.g., an Fc domain joined to a Jagged1, a growth factor, or a cytokine polypeptide or fragments thereof), HGF, and/or VEGF or other growth factors are useful for preventing or ameliorating tissue damage associated with reperfusion injury, hypofusion, ischemic injury, and/or low/no-reflow (e.g., a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like). In embodiments, the hypofusion is cerebral hypofusion, tissue hypofusion, and/or organ hypofusion. In one therapeutic approach, an isolated complex containing FGF2 and a fusion protein containing an Fc domain, or fragments thereof, optionally where the complex further contains VEGF, HGF, or fragments thereof, is administered systemically. The dosage of the administered isolated complex depends on a number of factors, including the size and health of the individual patient. For any particular subject, the specific dosage regimes should be adjusted, as necessary over time according to the individual need and the professional judgment of the person administering or supervising the administration of the compositions.

In embodiments, the composition is administered to a subject following myocardial ischemia with reperfusion. In embodiments, the composition is administered within about 1 hr, 2 hrs, 3 hrs, 4 hrs, 5 hrs, 6 hrs, 12 hrs, 24 hrs, 48 hrs, 72 hrs, 1 week, 2 weeks, 3 weeks, or 1 month of myocardial ischemia with reperfusion. In embodiments, the composition is administered within about 1 hr, 2 hrs, 3 hrs, 4 hrs, 5 hrs, 6 hrs, 12 hrs, 24 hrs, 48 hrs, 72 hrs, 1 week, 2 weeks, 3 weeks, or 1 month of a myocardial infarction. In embodiments the administration is within the indicated periods before and/or after myocardial ischemia with reperfusion or a myocardial infarction.

### Pharmaceutical Compositions

In one embodiment, a composition of the invention comprises or consists essentially of isolated complexes containing a fusion protein containing an Fc domain (e.g., an Fc domain joined to a Jagged1, a growth factor, or a cytokine polypeptide or fragments thereof), or fragments thereof, and FGF2, VEGF, and/or HGF, or fragments thereof. An isolated complex can be conveniently provided to a subject as sterile liquid preparations, e.g., isotonic aqueous solutions, suspensions, emulsions, dispersions, or viscous compositions, which may be buffered to a selected pH. A composition comprising isolated complexes containing FGF2 and a fusion protein containing an Fc domain, or fragments thereof, optionally where the complexes further contain VEGF, HGF, or fragments thereof, may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by mixing isolated complexes containing FGF2 and a fusion protein containing an Fc domain, or fragments thereof, optionally where the complexes further contain VEGF, HGF, or fragments thereof, in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and/or lachrymal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

Compositions comprising isolated complexes containing FGF2 and a fusion protein containing an Fc domain, or fragments thereof, optionally where the complexes further contain VEGF, HGF, or fragments thereof, are administered in an amount required to achieve a therapeutic or prophylactic effect. Such an amount will vary depending on the conditions. Typically, biologically active isolated complexes will be purified and subsequently concentrated so that the protein content of the composition is increased by at least about 5-fold, 10-fold, or 20-fold over the amount of protein originally present in the media. In other embodiments, the protein content is increased by at least about 25-fold, 30-fold, 40-fold or even by 50-fold. Preferably, the composition comprises an effective amount of isolated complexes containing FGF2 and a fusion protein containing an Fc domain, or fragments thereof, optionally where the complexes further contain VEGF, HGF, or fragments thereof.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit vasoprotection, carioprotection, reduce vascular injury, or induce another desirable biological response. Such determinations do not require undue experimentation, but are routine and can be ascertained without undue experimentation

### Methods of Delivery

Compositions comprising isolated complexes containing a fusion protein containing an Fc domain (e.g., an Fc domain joined to a Jagged1, a growth factor, or a cytokine polypeptide or fragments thereof), or fragments thereof, and FGF2, VEGF, and/or HGF, or fragments thereof, may be delivered to a subject in need thereof. The compositions may be delivered as part of a standard of care procedure. Modes of administration include intramuscular, intra-cardiac, oral, rectal, topical, intraocular, buccal, intravaginal, intracisternal, intra-arterial, intracerebroventricular, intratracheal, nasal, transdermal, within/on implants, e.g., fibers such as collagen, osmotic pumps, or parenteral routes. The term “parenteral” includes subcutaneous, intravenous, intramuscular, intraperitoneal, intragonadal or infusion. In instances, administration of a complexes of the invention is associated with a long-term increase in cardiac perfusion.

The compositions can be administered via localized injection, including catheter administration, systemic injection, localized injection, intravenous injection, or parenteral administration. When administering a therapeutic composition of the present invention, it will generally be formulated in a unit dosage injectable form (solution, suspension, emulsion). Dosages can be readily adjusted by those skilled in the art (e.g., a decrease in purity may require an increase in dosage). Compositions of the invention can be introduced by injection, catheter, or the like. Compositions of the invention include pharmaceutical compositions comprising cellular factors of the invention and a pharmaceutically acceptable carrier. Administration can be autologous or heterologous.

In embodiments, the compositions are infused from an indwelling percutaneous coronary intervention (PCI) guide catheter. The compositions can be infused before, after, or during stenting and/or restoration of blood flow. In instances, agents of the present invention (e.g., complexes containing FGF2 and a fusion protein containing an Fc domain, or fragments thereof, optionally where the complexes further contain VEGF, HGF, or fragments thereof) are administered by intracoronary infusion, optionally from a percutaneous coronary intervention (PCI) guide catheter. It can be advantageous to administer the compositions of the invention to a coronary artery, optionally following myocardial infarction (MI).

### Methods for Evaluating Therapeutic Efficacy

In one approach, the efficacy of a treatment is evaluated by measuring, as a non-limiting example, vascular integrity. Such methods are standard in the art and are described herein (see, e.g., the Examples provided below). In particular, a method of the present invention, decreases vascular permeability by at least about 5%, 10%, 20%, 40%, 50%, 60%, 70%, 80%, 90%, 100%. In some instances, the method of the present invention provides significant myocardial salvage and/or reduces infarct size. In one embodiment, therapeutic efficacy is assessed by measuring a reduction in apoptosis. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, CA), the ApoBrdU DNA Fragmentation Assay (BIOVISION, Mountain View, CA), and the Quick Apoptotic DNA Ladder Detection Kit (BIOVISION, Mountain View, CA). In another embodiment, therapeutic efficacy is assessed by measuring cell proliferation (e.g., using a CyQUANT assay). In some instances, efficacy is measured using electrophysiological recordings (e.g., using a CoreMap high density electrode array or an electrocardiogram).

### Kits

The invention provides kits for the treatment or prevention of a condition associated with reperfusion injury, hypofusion, ischemic injury, and/or low/no-reflow (e.g. a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound (e.g., a military wound), and the like). In one embodiment, the kit includes a therapeutic or prophylactic composition containing an effective amount of medium (e.g. concentrated human epicardial derived cell-conditioned medium) that contains complexes containing a fusion protein containing an Fc domain (e.g., an Fc domain joined to a Jagged1, a growth factor, or a cytokine polypeptide or fragments thereof), or fragments thereof, and FGF2, VEGF, and/or HGF, or fragments thereof, in unit dosage form. In some embodiments, the kit comprises a sterile container which contains a therapeutic or prophylactic composition of medium; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, medium of the invention is provided together with instructions for administering the medium to a subject having or at risk of developing reperfusion injury, hypofusion, ischemic injury, and/or low/no-reflow or in need of reperfusion after myocardial ischemia. The instructions will generally include information about the use of the composition for the treatment or prevention of a condition associated with reperfusion injury, hypofusion, ischemic injury, and/or low/no-reflow (e.g., a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound, and the like). In other embodiments, the instructions include at least one of the following: description of the medium; dosage schedule and administration for treatment or prevention of a condition associated with reperfusion injury, hypofusion, ischemic injury, and/or low/no-reflow (e.g., a burn, diabetic retinopathy, grafted and/or bioengineered tissues, ischemic stroke/injury, myocardial infarction, organ injury, peripheral artery disease (PAD), sepsis-induced vascular injury, surgery (e.g., associated with organ transplantation), vascular injury, a wound, and the like) or symptoms thereof; precautions; warnings; indications; counter-indications; over dosage information; adverse reactions; animal pharmacology; clinical studies; and/or references, the treatment regime, reagents, equipment (test tubes, reaction vessels, needles, syringes, etc.) and standards for calibrating or conducting the treatment. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Localization of HGF/IgG Complexes in Myocardial Tissue 24 Hours Following Infusion from PCI Guide Catheter after MI/

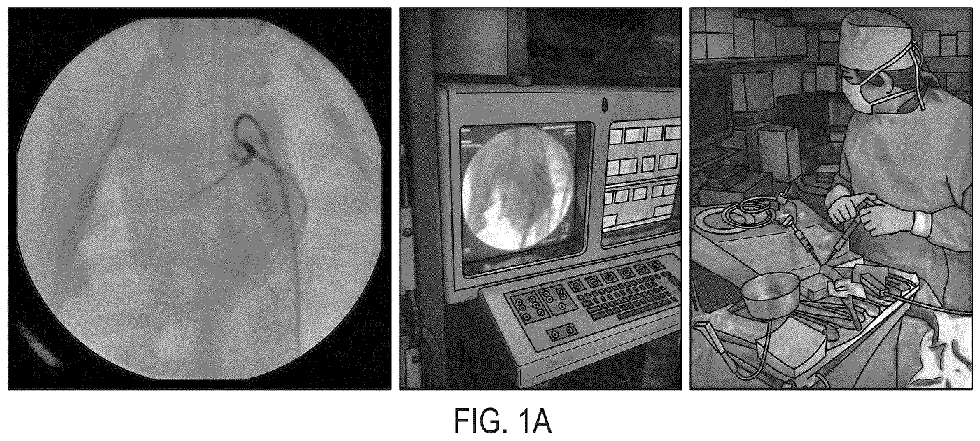

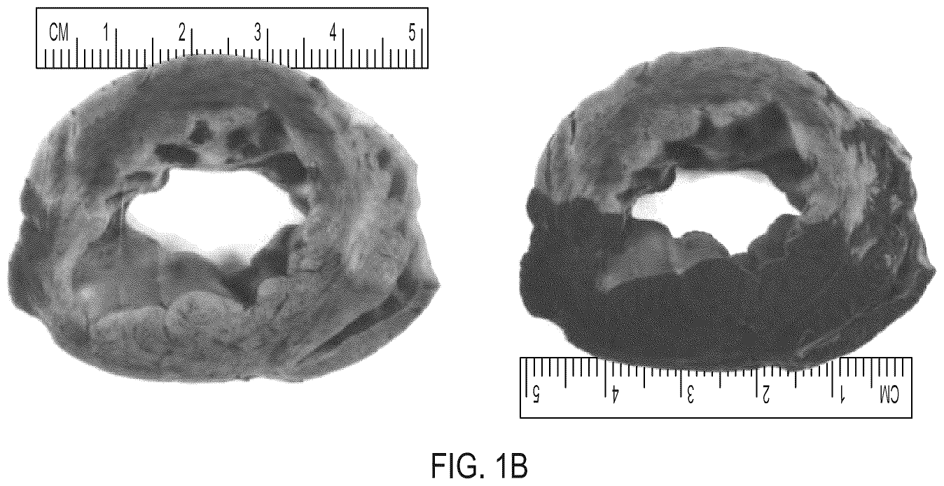

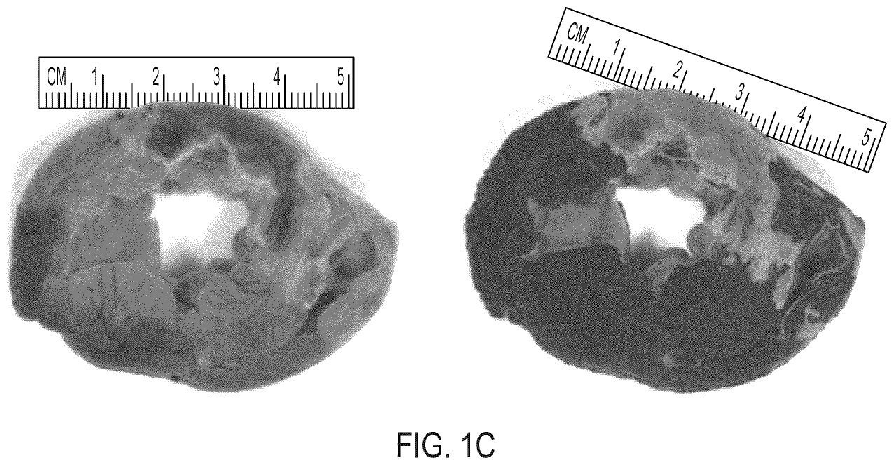

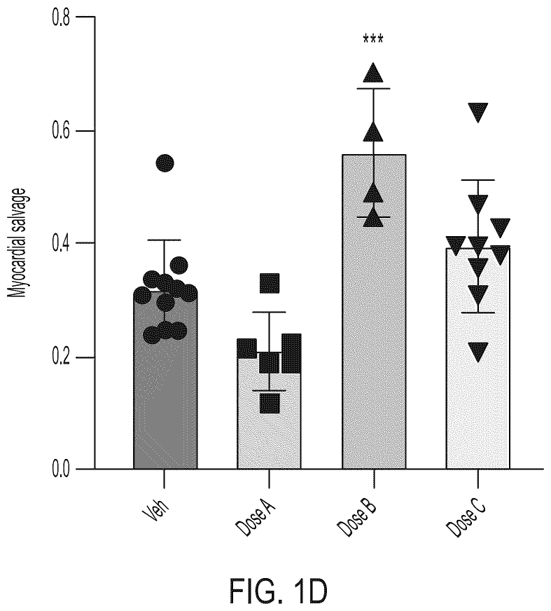

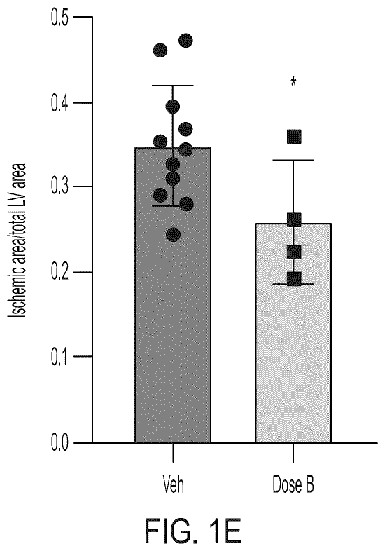

When administered IV, HGF has a plasma half-life of ˜3.8 min and is primarily taken up by the liver (Appasamy et al., 1993). To determine whether intracoronary delivery of human HGF/IgG complexes would improve retention and/or tissue distribution, as opposed to rapid clearance, MI/R surgery was performed in pigs. To mimic a clinical delivery scenario, a femoral artery access was used and PCI guide catheters, wires, angioplasty balloons and stents commonly used for interventional procedures at UVM Medical Center. Under fluoroscopic guidance, the catheters were advanced through the aorta and into the LAD (FIG. 1A). By injecting contrast dye, the relative degree of dominance was estimated for the ventricular blood supply coming from the left circumflex artery and the LAD. Accordingly, the balloon was placed over the guide wire at the first or second diagonal branch of the LAD, inflated, and then left inflated for 60 min to occlude the artery and produce ischemic injury. Immediately upon reperfusion, 12.5 mL of DMEM medium (vehicle) or 12.5 mL DMEM with HGF/IgG complexes was infused into the coronary from the indwelling guide catheter. The total time of infusion was ˜2 min.

After 24 hrs, pigs were intubated and re-anesthetized. Prior to euthanization, a suture was used to ligate the LAD at the proximal end of the stent and Evan's blue dye was infused to delineate areas of perfusion and risk (FIGS. 1B and 1C). Hearts were then removed, sliced at 1 cm thickness, and stained with TTC to identify areas of necrosis (FIGS. 1B and 1C). The sections were digitally-photographed and fixed in formalin for paraffin processing, sectioning and histology. Using Scion Image software, an observer blinded to sample/slide ID determined the area at risk (AAR) and ischemic area (IA), as well as total LV area. Hearts were assayed from pigs treated with 3 different doses of HGF/IgG, each matched at a 1:1 molar ratio. Compared with vehicle, a single intracoronary dose containing 62.5 μg of human HGF with 105 μg porcine IgG provided a significant degree of myocardial salvage (AAR-IA/AAR) at 24 hrs after MI/R and intracoronary treatment (FIG. 1D). The 62.5 μg HGF dose also significantly reduced infarct size, as shown by ischemic area (i.e. necrotic tissue)/total LV area (FIG. 1E).

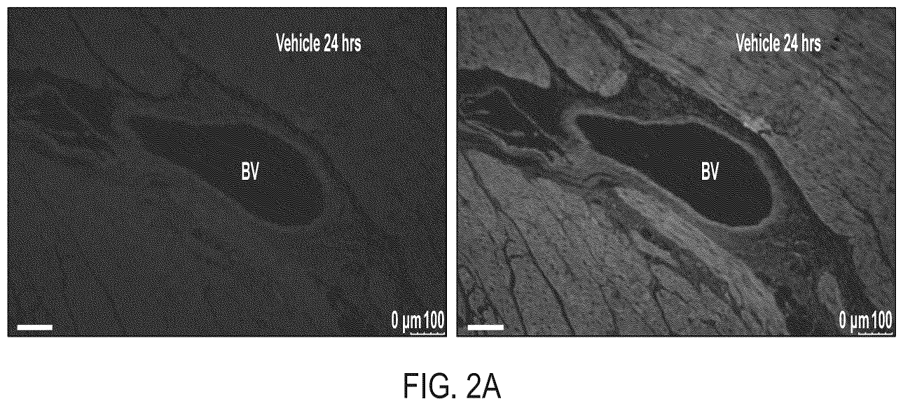

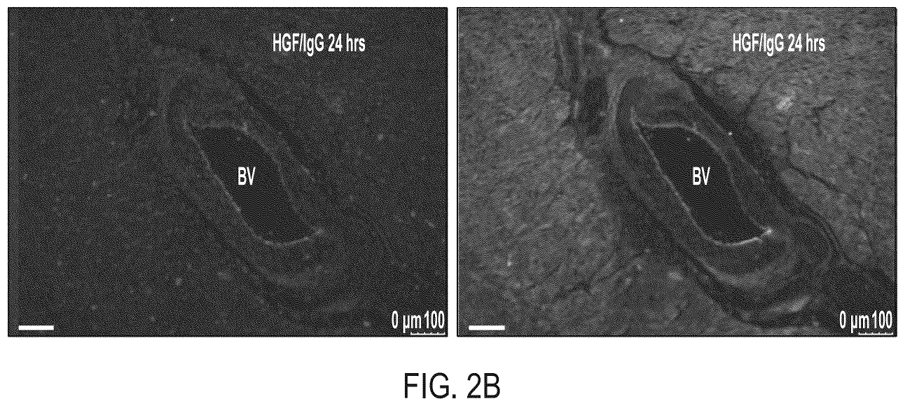

Immunohistochemistry was used with antisera against the 6×-His tag (SEQ ID NO: 16) on the recombinant human HGF to detect the HGF/IgG complexes in tissue sections (FIGS. 2A and 2B). Staining of left ventricular myocardial tissue bordering zones with infarction demonstrated a wide-spread distribution of HGF/IgG complexes; this pattern was associated with the microvasculature and arterioles as well as within myocardial tissue (n=3 pigs, FIG. 2B). As expected, similar staining of vehicle-treated control pigs did not identify any complexes and lacked the spotty pattern of complexes throughout the myocardium (n=3 pigs, FIG. 2A).

### Example 2: Multiple IV Infusions of HGF/IgG Complexes after MI do not Induce Production of Circulating HGF-Specific Auto-Antibodies

Autoimmune reaction is an important concern for clinical application of antibody-based biologics (Her and Kavanaugh, 2016). To examine the potential immunogenicity of HGF/IgG complexes in the context of MI with reperfusion, set of experiments were performed in Sprague Dawley rats to test for the production of auto-antibodies directed against HGF. Groups of adult rats underwent surgery to temporarily occlude the left anterior descending artery (LAD) for 2 hours, followed by reperfusion. With the use of echocardiography, cardiac injury was confirmed in all rats 1 week after MI, and M-mode images obtained at 2, 3, and 4 weeks after MI demonstrated decreased LV wall thickness, consistent with myocardial damage and necrosis.

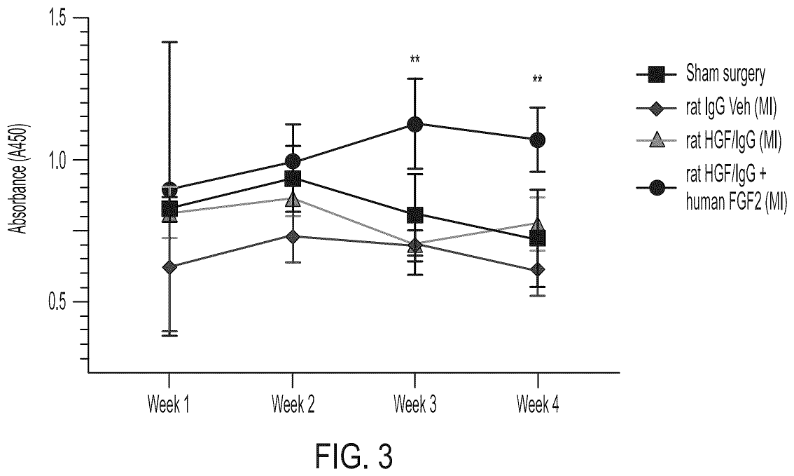

To form HGF/IgG complexes, recombinant rat HGF was mixed with polyclonal rat IgG and concentrated 40-fold by centrifugation with an ultracentrifugal filter unit. As a positive control for immune reactivity, one group of rats received rat HGF/rat IgG with recombinant human FGF2 added to the complexes. It was expected that multiple administrations with a xenogenic protein mixture would facilitate detection of circulating antibodies directed against rat HGF (i.e., auto-antibodies). Immediately after reperfusion, HGF/IgG complexes, HGF/IgG complexes with human FGF2, or IgG alone (vehicle control) were administered to 3 groups of rats. All treatments were infused directly into the left ventricle (LV) lumen through the apex (i.e., intra-arterial). Subsequently, all rats received two additional “booster” injections by tail vein on days 3 and 5 after MI. Blood plasma was obtained at weekly intervals post-surgery for 4 wks. Using a modified ELISA system, it was determined that animals treated with rat HGF/IgG (n=5) exhibited no autoimmune activity at any of the time points analyzed (FIG. 3). By contrast, compared with vehicle-treated controls, rats infused with rat HGF/IgG complexes and human FGF2 (n=5) exhibited a mild immune response at 3 wks and 4 wks following MI and treatment (FIG. 3). These results indicated that administered growth factor/IgG complexes are well-tolerated, particularly with a limited number of infusions and if species-matched for immune compatibility.

### Example 3: Identification of FGF2/IgG Complexes that Protect Human Cardiac Microvascular Endothelial Cells During Simulated Ischemia

Previously, it was demonstrated that a mixture of HGF and IgG (1:1 molar ratio) formed protein complexes after 40-fold concentration by centrifugation in Amicon ultracentrifugal filter units (Rao et al., 2015). To investigate whether FGF2 interacted non-covalently (e.g., electrostatically) with IgG, human FGF2 (LMW isoform, 18 kDa) was incubated with IgG in a 1:1 molar ratio mixture for 30 min at room temperature. Following 40-fold concentration with Amicon units, the samples were evaluated by native agarose gel electrophoresis as in Rao et al. (2015).

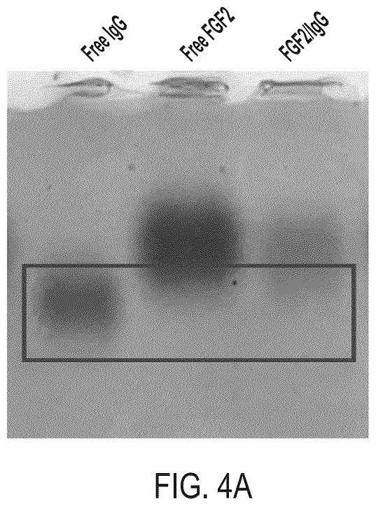

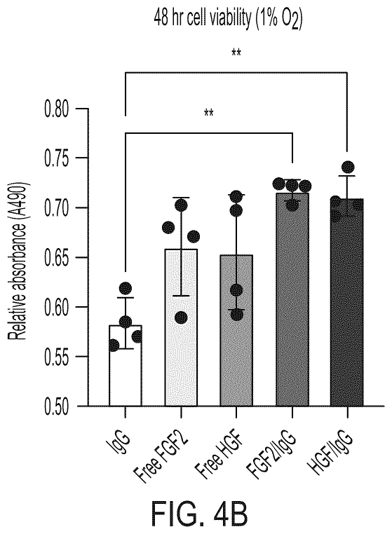

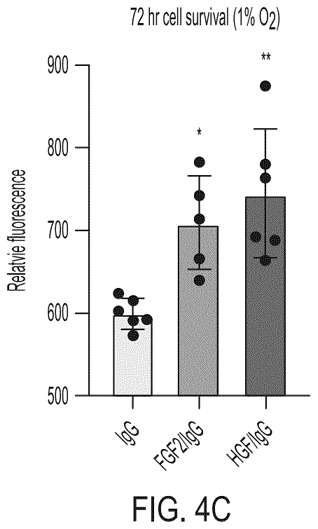

During native gel electrophoresis, protein separation occurred primarily by differences in isoelectric point rather than by size. After electrophoresis of free FGF2, free IgG, and FGF2/IgG protein samples in 1.3% agarose gels (50 mM MES buffer; pH, 7.0 to 8.5), staining with Coomassie Brilliant Blue dye demonstrated that the higher isoelectric point of FGF2 (pI=9.58) resulted in a slower migrating species compared with the migration pattern observed for free IgG (pI=6.6 to 7.2)(FIG. 4A). Of special interest, on gel lanes with concentrated FGF2/IgG mixtures, a distinct band shift was seen, whereby FGF2 reduced the migration distance for free IgG in the gel (FIG. 4A). A series of different gel runs demonstrated that the interaction of FGF2 with IgG occurred across a range of pH (7.0-8.5) (data not shown). Furthermore, by solid phase binding assays, a dose-responsive increase in low molecular weight FGF2 binding to IgG was observed (Table 1).

TABLE 1
Dose-responsive increase in human FGF2
binding to immobilized IgG. Numbers
represent mean absorbance (450 nm) and standard
deviation values from human FGF2 ELISA.
Assays were run in duplicate.

| Input: FGF2 | | |
|---|---|---|
| IgG | 4 ng/ml | 10 ng/ml |
| 100 ng/ml | 0.014 +/− 0.011 | 0.042 +/− 0.001 |
| 500 ng/ml | 0.04 +/− 0.003 | 0.071 +/− 0.006 |
| 10000 ng/ml | 0.193 +/− 0.148 | 0.46 +/− 0.045 |

FGF2/IgG was tested in cell protection assays with primary human cardiac microvascular endothelial cells under culture conditions that simulated ischemia (nutrient deprivation and 1% oxygen). By MTS assay, a measure of cell metabolism, FGF2/IgG complexes significantly improved endothelial cell viability during 48 hrs of exposure compared with cells that were incubated in a matched concentration of IgG (FIG. 4B). In agreement, quantification of cell survival using a nucleic acid dye-binding assay (Cyquant Direct) after 72 hrs of simulated ischemia demonstrated a significant increase in survival of microvascular endothelial cells incubated in FGF2/IgG complexes (FIG. 4C). Together, these assays indicated that FGF2/IgG complexes provided a similar level of microvascular endothelial cell protection as did HGF/IgG complexes, under matched experimental conditions (FIGS. 4B and 4C).

### Example 4: PatchDock/PyMOL Modeling of Human Growth Factor/IgG Interactions

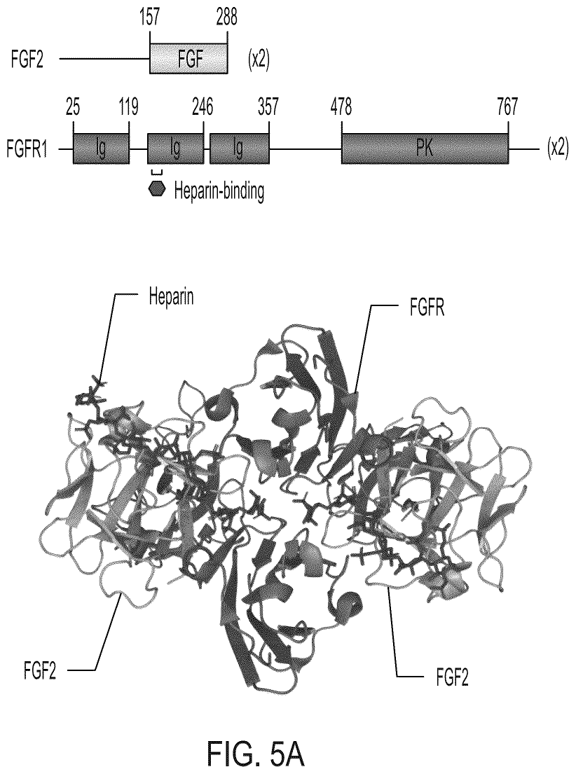

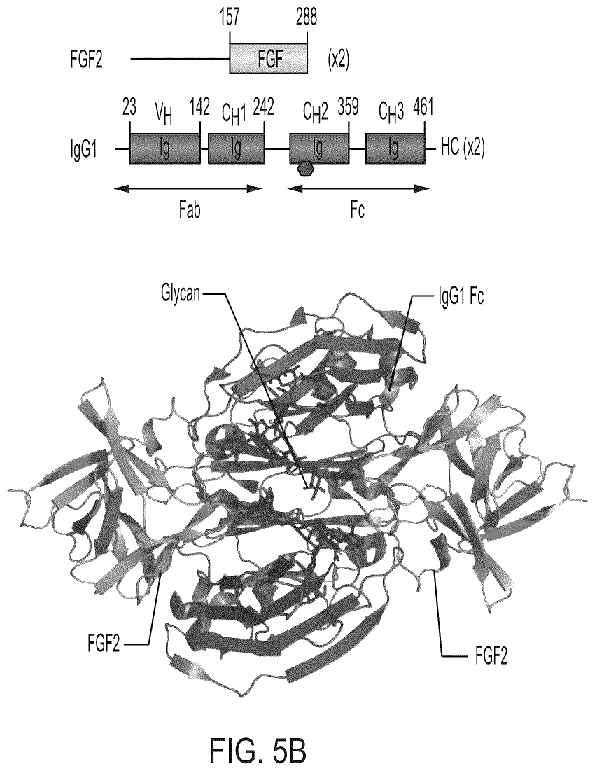

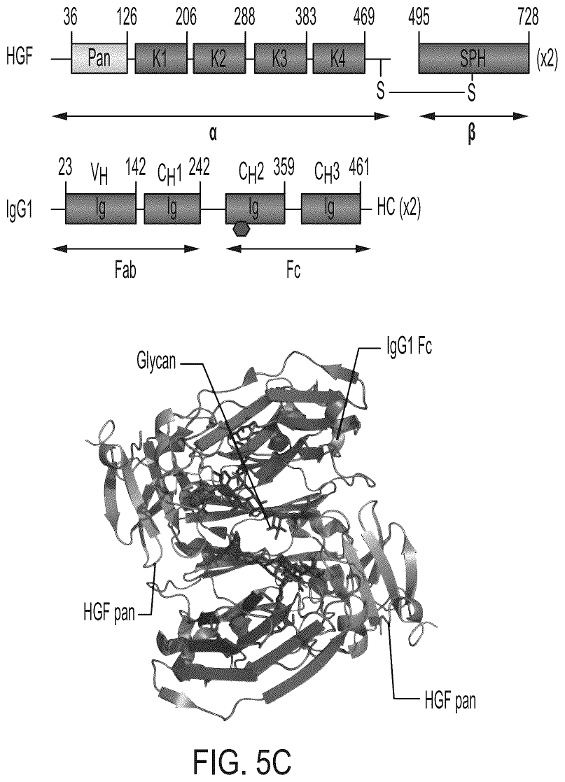

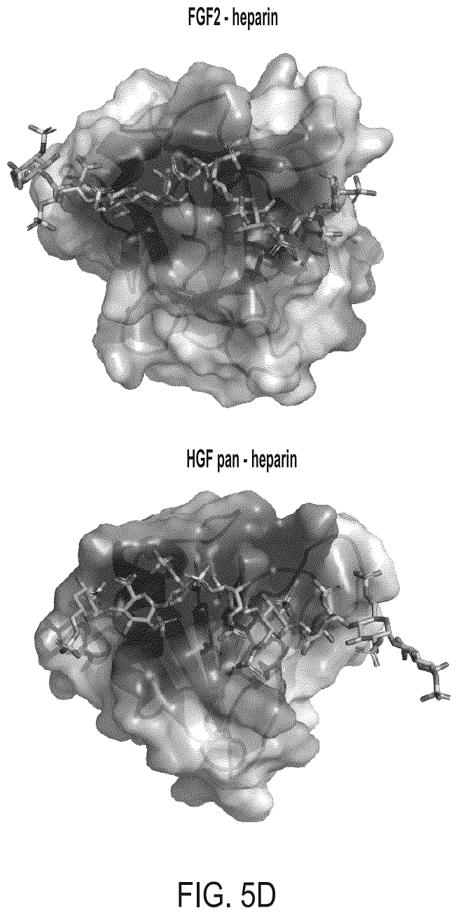

Given the dimeric binding of FGF2 to FGFR, experiments were undertaken to determine if FGF2/IgG complexes mimicked endogenous ligand-receptor binding and whether polyclonal IgG could structurally accommodate two FGF2 molecules. To ascertain the probable conformations of FGF2 and HGF in complex with IgG, 3-dimensional protein docking models were generated based on available crystal structures. The FGF2/IgG model was compared to a published crystal structure of FGF2 with heparin glycan co-factor bound to FGFR1 (FGFR1 PDB ID:1FQ9; FIG. 5A).

In docking experiments to model the geometry of FGF2/IgG complexes (FIG. 5B), the IgG1 crystal structure (PDB ID: 1HZH) was prepared in PyMOL, by removing waters and Fab fragments, leaving only the Fc dimer (residues 244-478 of 1HZH). This PDB file was uploaded to the PatchDock server as the “receptor” molecule. Next a single molecule of FGF2 (chain A from PDB ID 1CVS, residues 158-286 of the full-length 288 amino acid isoform 1) was uploaded as the “ligand” molecule. Clustering RMSD and complex type were both set to default before submitting the run. The top 100 solutions, scored based on surface geometric complementarity and area of contact, were retrieved. The first six high-scoring poses were eliminated as they placed the N- and/or C-termini of FGF2 facing into the surface of IgG. In this configuration, the full length FGF2 protein would extend from these residues and clash with IgG. Therefore, the seventh highest ranked pose was selected for use in the model (FIG. 5B).

A docking experiment to model possible interactions between HGF pan domain and IgG Fc was performed in a manner similar to that of the first docking experiment (FIG. 5C). First, IgG1 Fc (PDB ID: 1HZH with waters and Fab fragments removed) was uploaded as the “receptor” molecule. Then, the HGF pan domain (chain A from PDB ID: 3HMS, corresponding to residues 36-126 of the full-length 728 amino acid HGF isoform 1, was then uploaded to PatchDock as the “ligand” molecule, and the docking parameters were set as before. The top three poses were eliminated as in the first docking experiment due to clashes that would exist between full-length HGF and IgG. The fourth highest ranking pose was therefore selected for the model (FIG. 5C).

The majority of docking solutions placed FGF2 or HGF pan at the joint between the first and second Ig domains of the Fc region, with nearly an equal number on either of side of the Fc region of IgG in between the CH3 and CH2 domains.

The glycosylation sites on the CH3 Ig domain within the Fc portion of IgG were similar in position and size to the heparin-binding sites within the second IgG-like domain of FGFR. Thus, not intending to be bound by theory, it is plausible that IgG glycosylation may function in a manner analogous with heparin. In agreement with this concept, electrostatic surface potential models generated using the APBS plugin for PyMOL of the growth factor/IgG complexes highlighted the potential for interactions at heparin binding domains (FIG. 5D).

### Example 5: Complexes Formed Between Human FGF2 and Jagged1-Fc

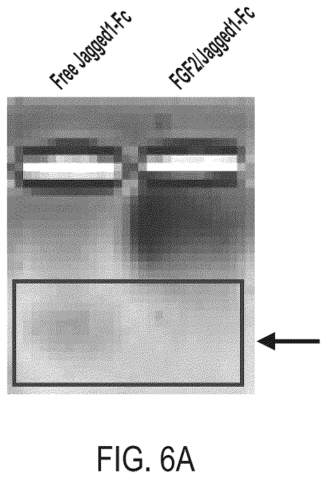

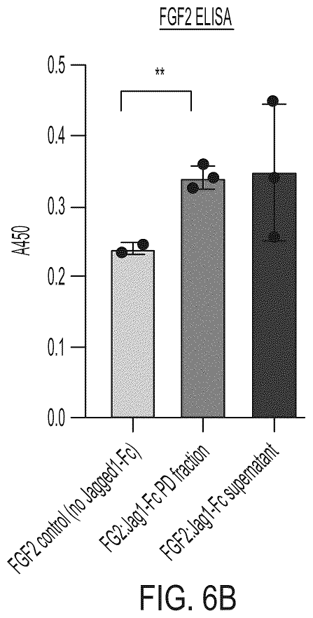

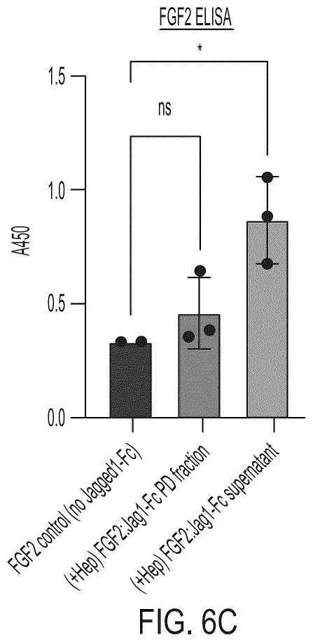

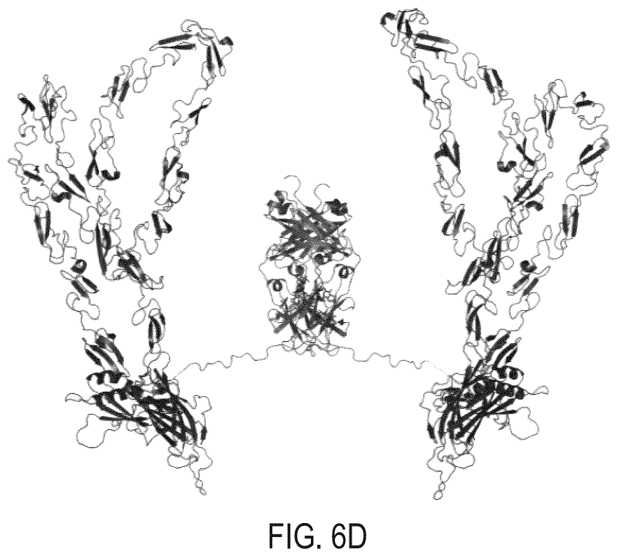

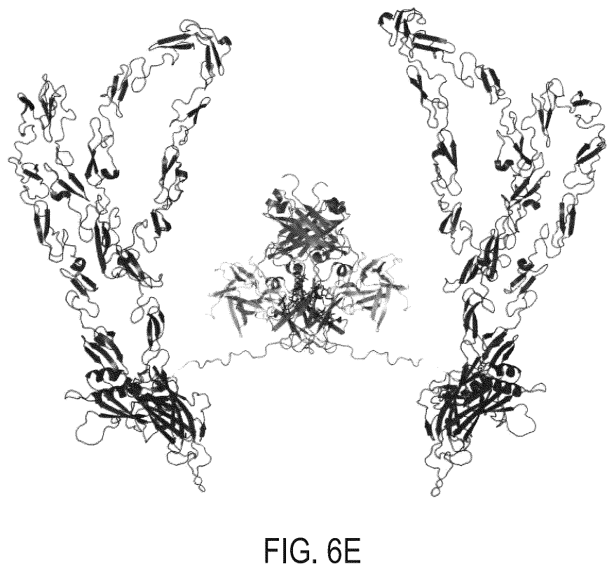

Experiments where next undertaken to determine whether fusion of the Fc domain from IgG1 to proteins of interest could be utilized to form new protein complexes. In particular, experiments were undertaken to determine whether an Fc domain would allow Jagged1, a Notch1 receptor ligand, to interact in a novel way with FGF2. Growth factors or cytokines that do not naturally bind IgG could potentially expressed the growth factors or cytokines as Fc-fusion proteins, and then be combined with HGF or FGF2 to form complexes. In support of the feasibility of this system, native gel electrophoresis of concentrated mixtures of FGF2/Jagged1-Fc demonstrated a shift in migration pattern relative to Jagged1-Fc alone (FIG. 6A).

To confirm complex formation with FGF2 and Jagged1-Fc, a series of biochemical pulldown experiments were performed. Taking advantage of the 6×histidine (SEQ ID NO: 16) purification tag on Jagged1-Fc, FGF2 and Jagged1-Fc were combined at an estimated equimolar ratio. Following a 2 hr incubation at room temperature and storage overnight at 4° C., the samples were incubated for 2 hr with anti-his-biotin. Then, streptavidin-agarose beads and centrifugation were used to pulldown the Jagged1-Fc. Following several washes, 0.5% Sodium Deoxycholate was used to dissociate the putative complexes and FGF2 ELISA was used to detect FGF2 that had complexed with Jagged1-Fc. By FGF2 ELISA, significantly more FGF2 was detected in pulldowns where FGF2 was incubated with Jagged1-Fc relative to control pulldowns, which contained FGF2 but lacked Jagged1-Fc (FIG. 6B). In support of the hypothesis that the Fc domain of IgG1 interacts with FGF2 in a manner similar to heparin, addition of excess heparin during incubation of FGF2 and Jagged1-Fc prevented complex formation; this resulted in FGF2 remaining in the supernatant rather than being pulled down by streptavidin-agarose (FIG. 6C).

To visualize a mode of Jagged1-Fc interaction with FGF2 consistent with our FGF2/IgG Fc docking results, a model was built of Jagged1-Fc fusion protein (R&D catalog #1277-JG) using the AlphaFold v2.0 human Jagged-1 model (Ser32-Ser1046) spliced to human IgG1 (residues corresponding to Pro100-Lys330 of Uniprot accession #P01857 of the 1HZH structure), via an Ile-Glu-Gly-Arg-Met-Asp linker (SEQ ID NO: 1) using PyMOL (Senior et al., 2020) (FIGS. 6D and 6E). The native gel and pulldown results, and models, illustrated how an Fc fusion protein and FGF2 can be combined to form unique protein complexes. Notably, this strategy provides a customizable platform to create new biologic drugs.

The results in adult pigs demonstrated that intra-arterially administered HGF/IgG complexes distributed throughout the myocardium after PCI and stenting and were retained for at least 24 hrs after MI/R. Of 3 intracoronary HGF doses tested, it was found that the middle dose with 62.5 μg human HGF and 105 μg porcine IgG was most effective at providing myocardial salvage and reducing infarct size. Furthermore, multiple infusions of species-matched HGF/IgG complexes after MI/R did not induce an immune response against HGF. Thus, as a formulation, HGF/IgG complexes can enhance retention of HGF and the duration of cytoprotective receptor signaling.

HGF is composed of α-subunit and β-subunits that are joined by a disulfide linkage. Functional domains within the subunits include an N-terminal hairpin domain, four kringle domains, and a catalytically inactive serine protease domain (Niemann, 2013). The canonical HGF receptor c-MET is a heterodimeric protein composed of a 50 kD extracellular α-chain and a 145 kD β-chain. In addition to the extracellular region, the β-chain also contains a single-pass transmembrane domain and a cytoplasmic tail which contains the active tyrosine kinase domain (Organ & Tsao, 2011). Analysis of crystal structures of HGF/c-MET showed HGF binding occurs within the β-chain, which contains a heparin sulphate binding domain (Organ & Tsao, 2011). HGF induces dimerization of c-MET, followed by tyrosine kinase activity and autophosphorylation, which triggers the recruitment of downstream adaptor molecules. Signaling through the HGF/c-MET axis results in signal propagation through multiple downstream adaptor pathways including: PI3K/Akt, Ras/Raf/MAPK, JAK/STAT and Wnt/0-Catenin (Holland et al., 2013; Koraishy, Silva, Mason, Wu, & Cantley, 2014; Organ & Tsao, 2011; Zhang et al., 2018). HGF signaling regulates tissue growth and morphogenesis during development, as well as cell migration, survival, angiogenesis and fibrosis during repair/remodeling after injury (Bottaro et al., 1991).

In a rat model of MI, Ono et al. reported elevated plasma levels of HGF were present 60 minutes following reperfusion (Ono, Matsumori, Shioi, Furakawa, & Sasayama, 1997). HGF and c-MET mRNA levels increased in the heart 3-fold at 24 to 48 hours following reperfusion, and remained elevated for 120 hours. Nakamura et al. reported treatment with recombinant HGF was cardioprotective against ischemia/reperfusion injury in rats (Nakamura et al., 2000), and reduced cardiomyocyte death and infarct size compared with controls. In contrast, administration of neutralizing antisera against endogenous HGF resulted in increased cardiac cell death and infarct expansion (Nakamura et al., 2000).

FGF2 has multiple isoforms ranging from 18 to 34 kDa that enable a wide array of possible quaternary structures that affect protein function (Yu, Ferrari, Galloway, Mignatti, & Pintucci, 2007). Several high molecular weight isoforms of FGF2 promote fibrosis after tissue injury. By contrast, low molecular weight FGF2 (18 kDa) and HGF are both reported to reduce fibrosis (Wang et al., 2004; Liao et al., 2007; Koo et al., 2018). Fibroblast Growth Factor Receptors (FGFRs) are tyrosine kinases activated by the binding of FGF ligands and heparan sulphate proteoglycans (HSPGs), which act as co-factors (Yayon, Klagsbrun, Esko, Leder, & Ornitz, 1991). The presence of HSPGs potentiates FGFR activity and also enhances ligand binding via a high affinity binding site (Ornitz & Itoh, 2015). HSPGs exist as cell surface-bound proteoglycans, transmembrane proteoglycans, or, are bound by the extracellular matrix (Matsuo & Kimura-Yoshida, 2013). FGF2-FGFR binding results in signal transduction through multiple pathways including RAS-MAPK, PI3K-AKT, PLCγ and STAT (Ahmad, Iwata, & Leung, 2012; Eliceiri, 2001; Liao et al., 2007a). FGF2 signaling plays fundamental roles in skeletal and neural development and controls the proliferation, differentiation, and survival of numerous adult somatic cell types. Also, it regulates critical processes such as angiogenesis and fibrosis during tissue remodeling/repair (Karajannis et al., 2006; Kardami et al., 2007; Müller, Meyer, & Werner, 2012).

In a canine model of MI, intracoronary injection of FGF2 was shown to reduce infarct size and improve cardiac function (Yanagisawa-Miwa et al., 1992). Furthermore, FGF2 treatment has been shown to promote myocardial angiogenesis, and result in increased vessel density in the FGF2-treated group compared with vehicle-treated control animals (Yanagisawa-Miwa et al., 1992). In isolated mouse hearts subjected to 60 min ischemia/reperfusion, specific knockout of low molecular weight FGF2 reduced capacity for recovery and cardiac performance relative to that of wild type mice (Liao et al., 2007a).

Intra-arterially administered FGF2 has been tested in clinical trials for patients with atherosclerotic peripheral arterial disease and intermittent claudication (Lazarous et al., 2000) as well as cardiac atherosclerosis (Simons et al., 2002; Lederman et al., 2002). Although one or two treatments with FGF2 was well-tolerated, efficacy was limited. For example, in the phase II FGF Initiating RevaScularization Trial (FIRST), a double-blind, randomized, and controlled study, a single intracoronary infusion of FGF2 was given to patients with severe coronary artery disease (Simons et al., 2002). Notably, however, treatment with FGF2 at 0, 0.3, 3, or 30 μg/kg did not improve exercise tolerance, the primary clinical endpoint (Simons et al., 2002). Lack of success in several trials was attributed, in part, to unsatisfactory pharmacokinetic profiles for FGF2. Similar to other growth factor proteins, FGF2 is quickly eliminated from the circulation, with a serum half-life of 50 min and poor target tissue distribution (Lazarous et al., 1997). Efforts directed at improving the pharmacokinetic profile of FGF2 may increase its therapeutic potential. For example, treatment with FGF2 encapsulated into sustained-release heparin-alginate coated capsules during coronary bypass graft surgery resulted in improved myocardial perfusion and a near complete cessation of angina related symptoms (Ruel et al., 2002).

The fusion of the Fc domain of IgG to proteins prolongs serum half-life, reduces clearance, and has been widely adopted for the production of therapeutic biologics. For example, Etanercept, an FDA-approved tumor necrosis factor receptor:Fc fusion protein, is indicated for multiple inflammatory conditions such as rheumatoid arthritis and plaque psoriasis (Scott, 2014). The improved pharmacokinetic profile of Fc fusion proteins is primarily due to Fc domain binding to the neonatal receptor for IgG (FcRn) (Czajkowsky, Hu, Shao, & Pleass, 2012; Roopenian & Akilesh, 2007). Similar to Fc-fusion proteins, the distribution and retention of HGF/IgG and FGF2/IgG complexes in the heart and other target tissues may benefit from interactions with FcRn.

The pro- and anti-inflammatory activities of IgGs are modulated through interaction of the IgG Fc domain with distinct IgG Fc receptors (FcγRs) on cells (Nimmerjahn & Ravetch, 2008). Heavy chain glycosylation of IgG Fc domains determines, in part, whether antibodies can bind FcγRs on immune cells to stimulate antibody-dependent cellular cytotoxicity (Shinkawa et al., 2002). Previous work suggests that IgG Fc core glycosylation, predominantly with sialic acid sugar moieties, may act as an “immunological switch”, imparting anti-inflammatory properties to IgGs (Anthony & Ravetch, 2010; Kaneko, Nimmerjahn, & Ravetch, 2006).

Extensive progress in the areas of biomaterials and bioengineering has improved growth factor delivery to tissues/organs with the purpose of promoting tissue repair and regeneration. Examples include scaffolds composed with biodegradable hydrogels or other substrates and seeded with paracrine-acting, reparative cells or purified growth factors. Diverse permutations and designs are possible, and hydrogels may be further modified with extracellular matrix components, drugs, microparticles or nanoparticles (Xu et al., 2018; Dong et al., 2020 Bruggeman et al., 2019; Roy et al., 2021). Whereas a systemic, intra-arterial route was used in the above Examples to administer growth factor/IgG complexes, therapeutic complexes can also be embedded within bioengineered substrates or matrices to control timing and localization of release for different applications. In addition to treating MI, growth factor/IgG complexes or FGF2/Fc-fusion complexes can be suited to improve tissue survival and function after organ transplantation or to treat other forms of injury such as diabetic neuropathy, peripheral artery disease, chronic ulcer, and stroke.

### Methods of the Examples

The following methods were employed in the above examples.

### Purification of Recombinant Human Growth Factors

To facilitate cell adhesion, 150 mm² plates (Nunc, Denmark) were pre-coated with human fibronectin (5 μg/mL in PBS). For production of recombinant human HGF, a stable HEK293 HGF-producer cell line (Rao et al., 2015) was grown in DMEM/F12 medium supplemented with 5% FBS (Atlanta Biologicals, Lawrenceville, GA, USA). Medium was changed every 2 days until the cells reached confluence, after which the media was switched to serum-free DMEM/F12. Cell-conditioned medium (Cdm) was collected after 24 and 48 hrs. To remove cell debris, Cdm was filtered (0.2 micron) and stored at −80° C. For purification by SP-Sepharose cation exchange chromatography, the Cdm was adjusted to pH 6.5 to facilitate HGF binding to resin. Cdm was loaded onto a SP-sepharose column that was prepared with an equilibration buffer: 20 mM sodium phosphate (pH 6.5) buffer with 0.02% Tween-80. The column was washed with 5 volumes of equilibration buffer and then 5 volumes of equilibration buffer with 0.05 M NaCl. The bound HGF was eluted using a two-step elution: 1) Low salt [equilibration buffer with 0.4 M NaCl], and 2) High salt [equilibration buffer with 0.8 M NaCl]. Peak fractions were determined by Bradford protein assay and human HGF ELISA according to the manufacturer's instructions (HGF Duoset kit, R&D systems).

Peak fractions eluted off SP-sepharose resin were pooled and diluted 1:1 with equilibration buffer: 20 mM sodium phosphate, 300 mM sodium chloride and 10 mM imidazole, pH 7.4. Fractions were then loaded by gravity onto a column with 2 mL of Ni-NTA resin (ThermoFisher). The column was washed with PBS supplemented with 25 mM imidazole, (pH 7.4) and eluted with PBS with 300 mM imidazole.

Recombinant human FGF2 (18 kDa) was purified from a bacterial expression system (E. coli strain BL21), as reported previously (Miao et al., 2020). Briefly, bacteria were grown in Luria-Bertani (LB) broth with ampicillin. Cells were lysed by sonication and the cell extract was pelleted (3,600×g for 10 min). The supernatant was loaded onto a 1 mL column with Ni-NTA resin (ThermoFisher). FGF2 was eluted with 400 mM imidazole. Protein concentration was determined by Bradford protein assay and protein composition and purity was assessed by SDS-PAGE. The eluate was desalted and loaded onto a 1 mL heparin-sepharose affinity column (GE Healthcare Bio-Sciences, Pittsburgh, PA, USA). Bound FGF2 was eluted from the heparin-sepharose column with PBS supplemented with 1.5 M NaCl.

### Human Endothelial Cell Protection Studies Under Simulated Ischemia

Primary human cardiac microvascular endothelial cells (HCMVEC) were from multiple donors and three vendors (Lonza Bioscience, Basel, Switzerland; PromoCell, Heidelberg, Germany; ScienCell, Carlsbad, CA, USA). HCMVEC cells were grown in endothelial cell-specific media with supplements (EGM-2MV SingleQuots, Lonza Bioscience; ECGS, PromoCell; ECGS, ScienCell) containing 5% FCS and PenStrep, at 37° C. with 5% CO2. Cells were seeded in tissue culture plates (Nunc), expanded as needed and frozen down. For cell protection studies, HCMVEC were used between passages 2 and 4. Primary HCMVECs were plated into 48-well plates (Nunc) at 1×10⁴ or 2×10⁴ cells/well. Cells were grown at 37° C. with 5% CO2 in endothelial cell growth medium with 5% FCS. All cells were plated 48 hrs prior to the start of the experiment. For simulated ischemia (oxygen and nutrient deprivation), cells were briefly washed with PBS to remove serum and the medium was switched to serum-free DMEM/F12 with or without various treatments. Cells were exposed to hypoxia (1% oxygen) in a dedicated, specialized incubator fed by nitrogen gas. After 48 or 72 hrs, plates were removed from the incubator, briefly rinsed with PBS, and frozen at −80° C. For MTS assay of cell viability, the CellTiter 96 Aqueous One solution Cell Proliferation Assay Kit (Promega) was used according to the manufacturer's instructions. For quantification of cell survival, a DNA binding dye was used (CyQuant assay, Molecular Probes Invitrogen). To ensure full cell lysis and dye incorporation, cells were subjected to 3× freeze-thaw cycles in lysis buffer. The lysis mixture was loaded into fluoroblock plates (Nunc) and relative fluorescence was determined by plate reader (Synergy HT, Biotek, Winooski, VT, USA).

### Preparation of Rat HGF/IgG Complexes

Recombinant rat HGF (Sino Biological US Inc., Wayne, PA, USA) was diluted to a working concentration of 2 μg/mL in sterile PBS. Non-specific, polyclonal IgG from rat serum (Sigma-Aldrich) was diluted to 1.2 μg/mL in sterile PBS. For each injection, a dose of 10 μg of HGF and 6 μg rat IgG was prepared. The HGF was mixed with the IgG in a total volume of 15 mL and concentrated 40-fold (from 15 mL to 375 μL over 45 min) using an Amicon ultracentrifugal filter unit (10 kDa cut-off filter; Millipore Sigma, Bedford, MA, USA).

Myocardial Ischemia-Reperfusion Surgery and Treatment with HGF/IgG Complexes in Rats

All animal work was approved by the University of Vermont College of Medicine's Office of Animal Care in accordance with the American Association for Accreditation of Laboratory Animal Care and National Institutes of Health guidelines. Sprague Dawley rats (8 wks of age) were anaesthetized with 4% isoflurane (to effect) and endotracheally-intubated. Rats were ventilated (Harvard Apparatus) and body temperature was maintained with a heated pad (Gaymar). Through a dermal incision, a blunt dissection was performed, and the intercostal muscles were separated. The heart was exposed, the left anterior descending coronary artery (LAD) was encircled with 10-0 nylon suture, and the LAD was occluded. The occlusion was confirmed by blanching of the anterior wall of the left ventricle. The incision was closed, and the animals were allowed to recover off the ventilator. After 2 hrs of ischemia, the rats were reintubated under anesthesia, ventilated and the chest wall was reopened. The hearts were exposed, and the suture was released. Reperfusion was confirmed by observing blood flow through the LAD and color returning to the previously blanched area. Immediately after reperfusion, or each rat, 125 μL of PBS containing either rat IgG or rat HGF/IgG complexes was injected through the left ventricle wall and into the LV lumen. After the injection, the chest wall was closed with 2-3 layers of suture. The rats with MI were returned to the vivarium for recovery and were kept for additional infusions and collection of blood plasma. Subsequently, the animals received tail vein “booster” injections with HGF/IgG or IgG on days 3 and 5 post-operation. For sham-operated animals, the chest wall was opened to visualize the intact pericardium (twice); this approach corresponded to that for the ischemia/reperfusion surgery. In sham-operated animals, the suture was passed under the LAD, but the artery was not ligated. No other surgical manipulations were carried out with the sham-operated animals.

### Modified ELISA for Auto-Antibodies Produced Against HGF/IgG Complexes

Following ischemia-reperfusion surgery, blood was drawn at 1, 2, 3 and 4 wk time points, centrifuged, and the resulting plasma was collected and stored at −80° C. For the detection of auto-antibodies against rat HGF in blood plasma, assays were performed as previously described (Gregory, Reyes, Whitney, & Spees, 2006; Spees et al., 2004). Briefly, 1 μg/mL recombinant rat HGF (Sino Biologics; Beijing, China) suspended in PBS was adsorbed onto high protein-binding plates (Nunc) overnight at 4° C., with shaking. The following day, the plate was blocked in PBS (pH 7.4) with 1% BSA (ThermoFisher Scientific) for 2 hrs at room temperature. Following blocking and additional washes, plasma samples were diluted (50-fold or 100-fold) in blocking buffer (1% BSA in PBS, pH 7.4, 0.2 μm filtered) and incubated overnight at 4° C., with shaking. The next day, the plasma wells were washed and incubated for 2 hrs with goat anti-rat horseradish peroxidase-conjugated antibodies (1:200, Invitrogen). A reference standard curve was created with a polyclonal rabbit anti-HGF antibody (Sigma-Aldrich) and pure rat HGF that was pre-adsorbed to the wells. The reference samples were washed and incubated 2 hrs with a goat anti-rabbit horseradish peroxidase-conjugated antibody (1:2000, Sigma-Aldrich). Following PBS washes, the plate was incubated with Ultra TMB substrate solution (ThermoFisher Scientific) for 50 min. Following color development, stop solution (2 M sulfuric acid) was added to neutralize the substrate solution and absorbance was determined at 450 nM (Synergy HT; Biotek, Winooski, VT).

### Preparation of HGF/IgG Complexes for Porcine Injections

HGF/IgG complexes were prepared 12-16 hours prior to injection and stored at 4-6° C. A dose of 31.25, 62.5, or 125 μg human HGF and 52.5, 105, or 210 μg mixed polyclonal pig IgG was used for each pig, representing a 1:1 molar ratio for each dose tested. Sterile growth factors and IgG were mixed in a total volume of 5 mL and added to an Amicon centrifugal concentration unit. The mixture was then centrifuged at 1,100×g for 30 min at room temperature. In some cases, complexes were centrifuged for an additional 5 min, until a final volume of 300 μL was reached. The complexes were then stored at 4° C. overnight. On the day of injection, complexes were reconstituted in sterile DMEM/F12 at a total volume of 12.5 mL.

Myocardial Ischemia-Reperfusion Surgery in Adult Pigs and Treatment with HGF/IgG Complexes

All work with pigs was performed under a UVM Institutional Animal Care and Use Committee-approved protocol and followed USDA guidelines. Commercial swine (Barrows, ˜50 kg) were pre-medicated with Meloxicam (0.2 mg/kg, PO) sedated with an injection of ketamine (15-20 mg/kg, IM) and atropine (0.05-0.5 mg/kg, IM), masked with isoflurane (5%), and intubated. Anesthesia was maintained with isoflurane (2.5%, inhaled). Intravenous catheters were placed in each ear for drug and fluid administration. A baseline transthoracic echocardiography (ECHO) was performed. Under sterile conditions, a cut-down was done to access the right femoral artery and a 6F vascular introducer was inserted. A blood sample was taken (20 mL) and heparin administered (300 μl/kg, IV) to prevent clotting during the catheterization procedure. A bolus of amiodarone (50 mg, IV) was given to reduce heart irritability and a bolus of fentanyl (0.05 mg/kg, IV) followed by a fentanyl infusion (0.05 mg/kg/hr, IV) was given to reduce the amount of isoflurane required for anesthesia. Under fluoroscopic guidance, a guide catheter was inserted and advanced into the opening of the left anterior descending (LAD) coronary artery. This was followed by a guide wire that was advanced into the distal LAD. A balloon catheter (with or without a stent) was advanced over the wire and positioned in the LAD. Stents were used to later identify the previous position of the balloon during inflation. An infusion of amiodarone was started (10 mg/kg, IV; over 30 min) to additionally reduce heart irritability. The balloon was inflated to eliminate blood supply to a region of the heart muscle for 60 minutes. Pigs were monitored by electrocardiogram and pulse-oximetry and provided with fluid and thermal support. In the event that ventricular tachycardia or cardiac arrest occurred during the occlusion, appropriate measures were taken to resuscitate the pig, including intravenous lidocaine, amiodarone, epinephrine, and electrical defibrillation.

The balloon was deflated, and reperfusion was confirmed by injection of a bolus of contrast dye through the guide catheter. DMEM/F12 (vehicle control) or protein complexes in vehicle were infused into the LAD from the indwelling guide catheter. Treatments were administered by manual injection in the LAD artery (a.k.a. intracoronary [12.5 ml]). Following occlusion, physiological parameters and cardiac arrest events were carefully recorded for all pigs. When present, ventricular fibrillation events were promptly addressed with DC cardioversion. The catheter was removed and the femoral artery blood flow restored. After the access site was repaired, additional pain medication was administered (Meloxicam; 0.02 mg/kg, SC), and the animals were monitored continuously until anesthesia wore off and then were transferred to a housing facility.

At 24 hrs after MI/R surgery and treatment, animals were re-anesthetized and a median sternatomy was performed to gain access to the heart. Umbilical tape snares (⅛ inch wide) were placed around the descending aorta, left subclavian artery, & bracheocephalic artery, but not occluded. A silk suture was placed around the LAD artery at the location of the previous balloon inflation, but not occluded. A 7F catheter was inserted into the right carotid artery and the tip advanced beyond the snare into the ascending aorta above the aortic valve. After ventricular fibrillation was induced by brief contact of the myocardium with a 9 V direct current battery, the LAD and snares were occluded. The isolated coronary circulation was perfused with 1.5% Evans blue via the carotid catheter to identify the area at risk. The heart was then removed, sliced at 1 cm thickness with a sharpened brain knife, and photographed to identify the area at risk. Slices were then incubated with 1.5% triphenyltetrazolium (TTC, 30° C.) for 25 min and photographed again to identify areas with infarction (white, necrotic).

### Immunohistochemistry:

Following Evans Blue/TTC staining of pig hearts, tissue slices were fixed in 10% formalin and paraffin embedding. Ten-micron serial sections were made from blocks of tissue bordering the zone with infarction. Prior to immunohistochemical staining, paraffin was removed by xylenes and alcohol (100% to 70%). Tissue sections were washed with PBS and antibody retrieval was performed using 20 μg/mL proteinase K (20 min at RT). Slides were blocked for 1 hour in PBS containing 5% normal goat serum and 0.1% Triton X-100, washed with PBS, and incubated at 4° C. overnight with primary antibody (anti-6× histidine (“6× histidine” disclosed as SEQ ID NO: 16), 1:1000, Catalog number MA 1-21315; Invitrogen). Following incubation with primary antibody, the slides were washed 3×5 min with PBS and incubated with secondary antibody for 1 hour at room temperature (1:1000, rabbit anti-mouse IgG conjugated with ALEXA 594; Invitrogen). After 3×5 min washes in PBS, slides were mounted with Vectashield containing DAPI (Vector Labs). Epifluorescence images were taken using a Leica DM6000B microscope equipped with a CCD camera and Leica imaging software.

### Native Gel Electrophoresis for Detection of FGF2/IgG and FGF2/Jagged1-Fc Protein Complexes

Native gel electrophoresis was performed using 50 mM MES sodium buffer with 1.3% agarose (ThermoFisher Scientific). Adjustments to pH were made, depending on experimental parameters. All gels were run using ice-cold buffer. Purified FGF2, Jagged1-Fc, or IgG were run individually (i.e. free) or as FGF2/IgG or FGF2/Jagged1-Fc complexes to assess their respective mobilities. Human FGF2 was cloned, expressed, and purified (Miao et al., 2020; Rao et al., 2015). Jagged1-Fc was commercial (R&D Systems). Depending on the molar ratios for different complexes, the total protein load for each lane varied between 3.0 μg and 4.0 μg. After completion of runs, the gels were stained for 30 min at room temperature with Coomassie Brilliant Blue dye and de-stained overnight in 10% acetic acid in deionized water.

### Solid Phase Binding Assays

For solid phase binding assays, polyclonal human IgG was incubated overnight at different concentrations in PBS on ELISA plates (100, 500, or 10,000 ng/mL). Following 3×PBS washes, low molecular weight (18 kDa) human FGF2 was incubated at 4 or 10 ng/mL for 2 hrs. After additional PBS washes, the amount of bound FGF2 was detected by ELISA (R & D Systems).

Pulldown of FGF2/Jagged1-Fc complexes using biotinylated anti-6× Histidine (“6× Histidine” disclosed as SEQ ID NO: 16) and streptavidin-conjugated agarose Growth factor complexes were prepared in 25 μL of PBS (pH 7.4) by mixing 0.5 μg human FGF2 with 7.8 μg of rat Jagged1-Fc-His (cat #599-JG, R&D Systems). Notably, these amounts represented an estimated 1:1 molar ratio. As a control, separate tubes were also incubated with 25 μL of PBS (pH 7.4) and 0.5 μg human FGF2 alone (i.e. no Jagged1-Fc). The samples were incubated for 2 hours at room temperature (with shaking) and the tubes were then placed at 4° C. overnight. The following day, 2 μg of a biotinylated anti-6×His antibody (“6×His” disclosed as SEQ ID NO: 16) (Invitrogen) was added to the complexes and incubated for 2 hrs with shaking at 4° C. The complexes were then incubated with streptavidin-agarose beads (ThermoFisher Scientific) for 48 hrs, with shaking at 4° C. The beads were pelleted by centrifugation for 10 min at 3000× g. The supernatant was reserved for ELISA and the pelleted beads were washed 4× with PBS prior to incubation with 0.5% Sodium Deoxycholate (SDC) to disrupt protein-protein interactions. The pulldown supernatant and the detergent soluble fractions were diluted and analyzed by ELISA (human FGF2 Duoset Kit, R&D systems), according to the manufacturer's instructions. In some cases, excess heparin sodium (50 μg) was added to the initial 25 μL incubation. To control for possible effects of SDC on ELISA, an equivalent amount of SDC was added to all samples prior to assay.

### Modeling Geometry of Growth Factor/Immunoglobulin Complexes

Docking simulations were made with Patchdock molecular docking server using published crystal structures of IgG, FGF2 and HGF. Separate docking experiments were performed to identify probable associations between: 1) FGF2 and the Fc domain of IgG1, and 2) HGF pan domain and the Fc domain of IgG1. Figures were generated using PyMOL molecular visualization software.

### Statistical Analysis

Statistical analysis was performed with GraphPad Prism software (version 6.0e). Values were expressed as means±SD unless indicated otherwise. Comparisons of data from individual control and treatment groups were made by unpaired Student's t-Test. For experiments comparing multiple treatment groups, One-way ANOVA was performed with post-hoc testing. For studies comparing multiple treatment groups across multiple time points, a Two-way ANOVA with repeated measures design was used. Values of P<0.05 were considered statistically significant.

### LIST OF REFERENCES

Ahmad, I., Iwata, T., & Leung, H. Y. (2012, April 1). Mechanisms of FGFR-mediated carcinogenesis. Biochimica et Biophysica Acta—Molecular Cell Research. Elsevier. doi.org/10.1016/j.bbamcr.2012.01.004
Anthony, R. M., & Ravetch, J. V. (2010, May). A novel role for the IgG Fc glycan: The anti-inflammatory activity of sialylated IgG Fes. Journal of Clinical Immunology. J Clin Immunol. doi.org/10.1007/s10875-010-9405-6
Appasamy, R., Tanabe, M., Murase, N., Zarnegar, R., Venkataramanan, R., Van Thiel, D. H., & Michalopoulos, G. K. (1993). Hepatocyte growth factor, blood clearance, organ uptake, and biliary excretion in normal and partially hepatectomized rats. Laboratory Investigation, 68(3), 270-276.
Bagai, A., Dangas, G. D., Stone, G. W., & Granger, C. B. (2014). Reperfusion strategies in acute coronary syndromes. Circulation Research. doi.org/10.1161/CIRCRESAHA.114.302744
Banquet, S., Gomez, E., Nicol, L., Edwards-Levy, F., Henry, J. P., Cao, R., . . . Brakenhielm, E. (2011). Arteriogenic therapy by intramyocardial sustained delivery of a novel growth factor combination prevents chronic heart failure. Circulation, 124(9), 1059-1069.
Barć, P., Antkiewicz, M., Śliwa, B., Fra̧czkowska, K., Guziński, M., Dawiskiba, T., . . . Skóra, J. P. (2020). Double VEGF/HGF Gene Therapy in Critical Limb Ischemia Complicated by Diabetes Mellitus. Journal of Cardiovascular Translational Research, 1-7. doi.org/10.1007/s12265-020-10066-9
Basilico, C., Amesano, A., Galluzzo, M., Comoglio, P. M., & Michieli, P. (2008). A high affinity hepatocyte growth factor-binding site in the immunoglobulin-like region of met. Journal of Biological Chemistry, 283(30), 21267-21277.
Bottaro, D. P., Rubin, J. S., Faletto, D. L., Chan, A. M. L., Kmiecik, T. E., Vande Woude, G. F., & Aaronson, S. A. (1991). Identification of the hepatocyte growth factor receptor as the c-met proto-oncogene product. Science, 251(4995), 802-804.
Bruggeman, K. F., Rodriguez A L, Parish C L, Williams R J, Nisbet D R. Temporally controlled release of multiple growth factors from a self-assembling peptide hydrogel. Nanotechnology. 2016 Sep. 23; 27(38):385102.
Bruggeman, K. F., Moriarty N, Dowd E, Nisbet D R, Parish C L. Harnessing stem cells and biomaterials to promote neural repair. Br J Pharmacol. 2019 February; 176(3):355-368.
Bush, M. A., Samara E, Whitehouse M. J., Yoshizawa C., Novicki D. L., Pike M., Laham R. J., Simons M., Chronos N. A. Pharmacokinetics and pharmacodynamics of recombinant FGF-2 in a phase I trial in coronary artery disease. J Clin Pharmacol. 2001 April; 41(4):378-85.
Caballero Aguilar L. M., Silva S. M., Moulton S. E. Growth factor delivery: Defining the next generation platforms for tissue engineering. J Control Release. 2019 Jul. 28; 306:40-58.
Chirgadze, D. Y., Hepple, J. P., Zhou, H., Andrew Byrd, R., Blundell, T. L., & Gherardi, E. (1999). Crystal structure of the NK1 fragment of HGF/SF suggests a novel mode for growth factor dimerization and receptor binding. Nature Structural Biology, 6(1), 72-79.
Czajkowsky, D. M., Hu, J., Shao, Z., & Pleass, R. J. (2012, October). Fc-fusion proteins: New developments and future perspectives. EMBO Molecular Medicine. Wiley-Blackwell. doi.org/10.1002/emmm.201201379
Duhovny, D., Nussinov, R., & Wolfson, H. J. Efficient Unbound Docking of Rigid Molecules. In Gusfield et al., Ed. Proceedings of the 2'nd Workshop on Algorithms in Bioinformatics (WABI) Rome, Italy, Lecture Notes in Computer Science 2452, pp. 185-200, Springer Verlag, 2002.
Dong, Y., Cui, M., Qu, J., Wang, X., Kwon, S. H., Barrera, J., . . . Gurtner, G. C. (2020). Conformable hyaluronic acid hydrogel delivers adipose-derived stem cells and promotes regeneration of burn injury. Acta Biomaterialia, 108, 56-66.
Eisen, A., Giugliano, R. P., & Braunwald, E. (2016). Updates on acute coronary syndrome: A review. JAMA Cardiology. doi.org/10.1001/jamacardio.2016.2049
Eliceiri, B. P. (2001, December 7). Integrin and growth factor receptor crosstalk. Circulation Research. Lippincott Williams and Wilkins. doi.org/10.1161/hh2401.101084
Garcia-Dorado, D., Ruiz-Meana, M., & Piper, H. M. (2009). Lethal reperfusion injury in acute myocardial infarction: Facts and unresolved issues. Cardiovascular Research. doi.org/10.1093/cvr/cvp185
Gregory, C. A., Reyes, E., Whitney, M. J., & Spees, J. L. (2006). Enhanced Engraftment of Mesenchymal Stem Cells in a Cutaneous Wound Model by Culture in Allogenic Species-Specific Serum and Administration in Fibrin Constructs. Stem Cells, 24(10), 2232-2243.
Her, M., & Kavanaugh, A. (2016, January 1). Alterations in immune function with biologic therapies for autoimmune disease. Journal of Allergy and Clinical Immunology. Mosby Inc. doi.org/10.1016/j.jaci.2015.10.023
Holland, J. D., Györffy, B., Vogel, R., Eckert, K., Valenti, G., Fang, L., . . . Birchmeier, W. (2013). Combined Wnt/β-Catenin, Met, and CXCL12/CXCR4 Signals Characterize Basal Breast Cancer and Predict Disease Outcome. Cell Reports, 5(5), 1214-1227.
Kaneko, Y., Nimmerjahn, F., & Ravetch, J. V. (2006). Anti-inflammatory activity of immunoglobulin G resulting from Fc sialylation. Science, 313(5787), 670-673.
Koo, H. Y., El-Baz L M, House S, Cilvik S N, Dorry S J, Shoukry N M, Salem M L, Hafez H S, Dulin N O, Ornitz D M, Guzy R D. Fibroblast growth factor 2 decreases bleomycin-induced pulmonary fibrosis and inhibits fibroblast collagen production and myofibroblast differentiation. J Pathol. 2018 September; 246(1):54-66.
Koraishy, F. M., Silva, C., Mason, S., Wu, D., & Cantley, L. G. (2014). Hepatocyte Growth Factor (Hgf) Stimulates Low Density Lipoprotein Receptor-related Protein (Lrp) 5/6 Phosphorylation and Promotes Canonical Wnt Signaling. Journal of Biological Chemistry, 289(20), 14341-14350.
Lazarous, D. F., Shou, M., Stiber, J. A., Dadhania, D. M., Thirumurti, V., Hodge, E., & Unger, E. F. (1997). Pharmacodynamics of basic fibroblast growth factor: route of administration determines myocardial and systemic distribution. Cardiovascular Research, 36(1), 78-85.
Liao, S., Porter, D., Scott, A., Newman, G., Doetschman, T., & Schultz, J. E. J. (2007). The cardioprotective effect of the low molecular weight isoform of fibroblast growth factor-2: The role of JNK signaling. Journal of Molecular and Cellular Cardiology, 42(1), 106-120.
Miao, T., Little, A. C., Aronshtam, A., Marquis, T., Fenn, S. L., Hristova, M., . . . Oldinski, R. A. (2020). Internalized FGF-2-loaded nanoparticles increase nuclear ERK1/2 content and result in lung cancer cell death. Nanomaterials, 10(4). doi.org/10.3390/nano10040612
Nakamura, T, Mizuno, S., Matsumoto, K., Sawa, Y., Matsuda, H., & Nakamura, T. (2000). Myocardial protection from ischemia/reperfusion injury by endogenous and exogenous HGF. The Journal of Clinical Investigation, 106(12), 1511-1519.
Nakamura, Toshikazu, & Mizuno, S. (2010, June). The discovery of Hepatocyte Growth Factor (HGF) and its significance for cell biology, life sciences and clinical medicine. Proceedings of the Japan Academy Series B: Physical and Biological Sciences. Proc Jpn Acad Ser B Phys Biol Sci. doi.org/10.2183/pjab.86.588
Nih, L. R., Gojgini, S., Carmichael, S. T., & Segura, T. (2018). Dual-function injectable angiogenic biomaterial for the repair of brain tissue following stroke. Nature Materials, 17(7), 642.
Nimmerjahn, F., & Ravetch, J. V. (2008, January). Fcγ receptors as regulators of immune responses. Nature Reviews Immunology. Nature Publishing Group. doi.org/10.1038/nri2206
O'Gara, P. T., Kushner, F. G., Ascheim, D. D., Casey, D. E., Chung, M. K., de Lemos, J. A., . . . Zhao, D. X. (2013). 2013 ACCF/AHA Guideline for the Management of ST-Elevation Myocardial Infarction. Journal of the American College of Cardiology. doi.org/10.1016/j.jacc.2012.11.019
Ono, K., Matsumori, A., Shioi, T., Furakawa, Y., & Sasayama, S. (1997). Enhanced expression of hepatocyte growth factor/c-Met by myocardial ischemia and reperfusion in a rat model. Circulation, 95(11), 2552-2558.
Organ, S. L., & Tsao, M. S. (2011). An overview of the c-MET signaling pathway. Therapeutic Advances in Medical Oncology. Ther Adv Med Oncol. doi.org/10.1177/1758834011422556
Ornitz, D. M., & Itoh, N. (2015). The fibroblast growth factor signaling pathway. Wiley Interdisciplinary Reviews: Developmental Biology, 4(3), 215-266.
Rao, K. S., Aronshtam, A., McElory-Yaggy, K. L., Bakondi, B., VanBuren, P., Sobel, B. E., & Spees, J. L. (2015). Human epicardial cell-conditioned medium contains HGF/IgG complexes that phosphorylate RYK and protect against vascular injury. Cardiovascular Research, 107(2), 277-286.
Roopenian, D. C., & Akilesh, S. (2007, September 17). FcRn: The neonatal Fc receptor comes of age. Nature Reviews Immunology. Nature Publishing Group. doi.org/10.1038/nri2155
Roy, T., James, B. D., & Allen, J. B. (2021). Anti-VEGF-R2 Aptamer and RGD Peptide Synergize in a Bifunctional Hydrogel for Enhanced Angiogenic Potential. Macromol Biosci. 21(2):e2000337.
Ruel, M., Laham, R. J., Parker, J. A., Post, M. J., Ware, J. A., Simons, M., & Sellke, F. W. (2002). Long-term effects of surgical angiogenic therapy with fibroblast growth factor 2 protein. Journal of Thoracic and Cardiovascular Surgery, 124(1), 28-34.
Schneidman-Duhovny, D., Inbar, Y., Nussinov, R., & Wolfson, H. J. (2005). PatchDock and SymmDock: servers for rigid and symmetric docking. Nucl. Acids. Res., 33: W363-367.
Schlessinger, J., Plotnikov, A. N., Ibrahimi, O. A., Eliseenkova, A. V., Yeh, B. K., Yayon, A., . . . Mohammadi, M. (2000). Crystal structure of a ternary FGF-FGFR-heparin complex reveals a dual role for heparin in FGFR binding and dimerization. Molecular Cell, 6(3), 743-750.
Schneider, D. J., & Sobel, B. E. (2008). A Novel Role for Tissue-Type Plasminogen Activator. Circulation.
Scott, L. J. (2014, August 1). Etanercept: a review of its use in autoimmune inflammatory diseases. Drugs. Springer. doi.org/10.1007/s40265-014-0258-9
Senior, A. W., Evans, R., Jumper, J. et al. (2020). Improved protein structure prediction using potentials from deep learning. Nature, 577, 706-710.
Shinkawa, T., Nakamura, K., Yamane, N., Shoji-Hosaka, E., Kanda, Y., Sakurada, M., . . . Shitara, K. (2002). The Absence of Fucose but Not the Presence of Galactose or Bisecting N-Acetylglucosamine of Human IgG1 Complex-type Oligosaccharides Shows the Critical Role of Enhancing Antibody-dependent Cellular Cytotoxicity*. doi.org/10.1074/jbc.M210665200
Simons, M., Annex, B. H., Laham, R. J., Kleiman, N., Henry, T., Dauerman, H., . . . Chronos, N. A. (2002). Pharmacological treatment of coronary artery disease with recombinant fibroblast growth factor-2: Double-blind, randomized, controlled clinical trial. Circulation, 105(7), 788-793.
Spees, J. L., Gregory, C. A., Singh, H., Tucker, H. A., Peister, A., Lynch, P. J., . . . Prockop, D. J. (2004). Internalized antigens must be removed to prepare hypoimmunogenic mesenchymal stem cells for cell and gene therapy. Molecular Therapy. Academic Press Inc. doi.org/10.1016/j.ymthe.2004.02.012
Spivak-Kroizman, T., Lemmon, M. A., Dikic, I., Ladbury, J. E., Pinchasi, D., Huang, J., . . . Lax, I. (1994). Heparin-induced oligomerization of FGF molecules is responsible for FGF receptor dimerization, activation, and cell proliferation. Cell, 79(6), 1015-1024.
Suzuki T., Ishii-Watabe A., Tada M., Kobayashi T., Kanayasu-Toyoda T., Kawanishi T., Yamaguchi T. Importance of neonatal FcR in regulating the serum half-life of therapeutic proteins containing the Fc domain of human IgG1: a comparative study of the affinity of monoclonal antibodies and Fc-fusion proteins to human neonatal FcR. J Immunol. 2010 Feb. 15; 184(4):1968-76.
Verma, S., Fedak, P. W. M., Weisel, R. D., Butany, J., Rao, V., Maitland, A., . . . Yau, T. M. (2002). Fundamentals of Reperfusion Injury for the Clinical Cardiologist. Circulation, 105(20), 2332-2336.
Virani, S. S., Alonso, A., Benjamin, E. J., Bittencourt, M. S., Callaway, C. W., Carson, A. P., . . . Heard, D. G. (2020, March 3). Heart disease and stroke statistics-2020 update: A report from the American Heart Association. Circulation. Lippincott Williams and Wilkins. doi.org/10.1161/CIR.0000000000000757
Vogel, B., Claessen, B. E., Arnold, S. V., Chan, D., Cohen, D. J., Giannitsis, E., . . . Mehran, R. (2019, December 1). ST-segment elevation myocardial infarction. Nature Reviews Disease Primers. Nature Publishing Group. doi.org/10.1038/s41572-019-0090-3
Xu Q., A. S., Gao Y., Guo L., Creagh-Flynn J., Zhou D., Greiser U., Dong Y., Wang F., Tai H., Liu W., Wang W., Wang W. A hybrid injectable hydrogel from hyperbranched PEG macromer as a stem cell delivery and retention platform for diabetic wound healing. Acta Biomater. 2018 Jul. 15; 75:63-74.
Yanagisawa-Miwa, A., Uchida, Y., Nakamura, F., Tomaru, T., Kido, H., Kamijo, T., . . . Ito, H. (1992). Salvage of infarcted myocardium by angiogenic action of basic fibroblast growth factor. Science, 257(5075), 1401-1403.
Yang, Z. J., Zhang, Y. R., Chen, B., Zhang, S. L., Jia, E. Z., Wang, L. S., . . . Wu, C. T. (2009). Phase i clinical trial on intracoronary administration of Ad-hHGF treating severe coronary artery disease. Molecular Biology Reports, 36(6), 1323-1329.
Yayon, A., Klagsbrun, M., Esko, J. D., Leder, P., & Ornitz, D. M. (1991). Cell surface, heparin-like molecules are required for binding of basic fibroblast growth factor to its high affinity receptor. Cell, 64(4), 841-848.
Yellon, D. M., & Hausenloy, D. J. (2007). Myocardial Reperfusion Injury. New England Journal of Medicine, 357(11), 1121-1135.
Yu, P. J., Ferrari, G., Galloway, A. C., Mignatti, P., & Pintucci, G. (2007). Basic fibroblast growth factor (FGF-2): The high molecular weight forms come of age. Journal of Cellular Biochemistry, 100(5), 1100-1108.
Wang, Y., Ahmad, N., Wani, M. A., Ashraf, M. (2004). Hepatocyte growth factor prevents ventricular remodeling and dysfunction in mice via Akt pathway and angiogenesis. J Mol Cell Cardiol. 37(5):1041-52.
Zhang, Y., Xia, M., Jin, K., Wang, S., Wei, H., Fan, C., . . . Xiong, W. (2018, February 19). Function of the c-Met receptor tyrosine kinase in carcinogenesis and associated therapeutic opportunities. Molecular Cancer. BioMed Central Ltd.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The invention may be related to Rao, K., et al. “Human epicardial cell-conditioned medium contains HGF/IgG complexes that phosphorylate RYK and protect against vascular injury”, Cardiovascular Res., 107:277-286 (2015); to Rao, Krithika, “Epicardial Cell Engraftment And Signaling Promote Cardiac Repair After Myocardial Infarction” (2016). Graduate College Dissertations and Theses. 479; to Liebman, et al. “Human growth factor/IgG complexes for treatment of myocardial ischemia-reperfusion injury,” Frontiers in Bioengineering and Biotechnology (Accepted: 26 Jan. 2022); to U.S. Provisional Patent Application No. 63/229,138, filed Aug. 4, 2021; or to U.S. Pat. No. 10,239,926 B2, to the entirety of which are incorporated herein by reference for all purposes.

What is claimed is:

1. An isolated complex comprising a growth factor polypeptide, or a fragment thereof, and a fusion protein comprising an immunoglobulin G (IgG) Fc polypeptide, or a fragment thereof, and a polypeptide of interest, or a fragment thereof.

2. The isolated complex of claim 1 wherein the growth factor polypeptide is selected from the group consisting of fibroblast growth factor (FGF2), hepatocyte growth factor (HGF), and vascular endothelial growth factor (VEGF).

3. The isolated complex of claim 1, wherein the polypeptide of interest is a growth factor or a cytokine.

4. The isolated complex of claim 1, wherein the polypeptide of interest is Jagged-1.

5. The isolated complex of claim 1, wherein the Fc polypeptide is separated from the polypeptide of interest by a peptide linker.

6. The isolated complex of claim 5, wherein the peptide linker comprises the amino acid sequence IEGRMD (SEQ ID NO: 1).

7. The isolated complex of claim 1, wherein the polypeptides are complexed by only non-covalent interactions and/or wherein the complex does not comprise an antibody-antigen interaction.

8. A composition comprising the complex of claim 1.

9. A pharmaceutical composition for increasing vascular integrity, promoting angiogenesis, increasing myocardial salvage, reducing infarct size, and/or preserving cardiac tissue, the composition comprising the complex of claim 1 and a pharmaceutically acceptable excipient.

10. A method for producing a complex, the method comprising contacting an isolated growth factor polypeptide or a fragment thereof with a fusion protein comprising an immunoglobulin G (IgG) Fc polypeptide, or a fragment thereof, and a polypeptide of interest, or a fragment thereof, thereby forming the complex.

11. The method of claim 10, wherein the growth factor polypeptide is selected from the group consisting of fibroblast growth factor (FGF2), hepatocyte growth factor (HGF), and vascular endothelial growth factor (VEGF).

12. The method of claim 11, wherein the polypeptide of interest is a growth factor or a cytokine.

13. The method of claim 11, wherein the polypeptide of interest is Jagged-1.

14. The method of claim 10, wherein the complex does not comprise an antibody-antigen interaction and the polypeptides of the complex are associated with one another by only non-covalent interactions.

15. The method of claim 10, wherein the isolated growth factor polypeptide or a fragment thereof is contacted with the fusion protein at a molar ratio of about 1:1.

16. A method for reducing cell damage or cell death following an ischemic event with reperfusion, the method comprising contacting a cell with the complex of claim 1, thereby reducing cell damage or cell death following the ischemic event with reperfusion.

17. The method of claim 16, wherein the ischemic event is associated with a myocardial infarction.

18. The method of claim 16, wherein the cell is a vascular endothelial cell, a vascular smooth muscle cell, a vascular or cardiac fibroblast, or a cardiac myocyte.

19. The method of claim 18, wherein the vascular endothelial cell is a microvascular endothelial cell.

20. A method for increasing vascular integrity, promoting angiogenesis, increasing myocardial salvage, reducing infarct size, and/or preserving tissue in a subject following an ischemic event with reperfusion, the method comprising administering to the subject the complex of claim 1, thereby increasing vascular integrity, promoting angiogenesis, increasing myocardial salvage, reducing infarct size, and/or preserving cardiac tissue relative to a reference.

21. A method for reducing vascular permeability in a subject following an ischemic event with reperfusion, the method comprising administering to the subject the complex of claim 1, thereby reducing vascular permeability relative to a reference.

22. The method of claim 20, wherein the ischemic event is associated with a myocardial infarction.

23. An isolated complex comprising fibroblast growth factor (FGF2), or a fragment thereof, and a fusion protein comprising an immunoglobulin G (IgG) Fc polypeptide, or a fragment thereof, and a Jagged-1 polypeptide, or a fragment thereof.

24. The isolated complex of claim 23, wherein the fusion protein has an amino acid sequence with at least 85% identity to the following sequence or a fragment thereof:
(SEQ ID NO: 2)
SGQFELEILSMQNVNGELQNGNCCGGARNPGDRKCTRDECDTYFK
VCLKEYQSRVTAGGPCSFGSGSTPVIGGNTFNLKASRGNDRNRIV
LPFSFAWPRSYTLLVEAWDSSNDTVQPDSIIEKASHSGMINPSRQ
WQTLKQNTGVAHFEYQIRVTCDDYYYGFGCNKFCRPRDDFFGHYA
CDQNGNKTCMEGWMGPECNRAICRQGCSPKHGSCKLPGDCRCQYG
WQGLYCDKCIPHPGCVHGICNEPWQCLCETNWGGQLCDKDLNYCG
THQPCLNGGTCSNTGPDKYQCSCPEGYSGPNCEIAEHACLSDPCH
NRGSCKETSLGFECECSPGWTGPTCSTNIDDCSPNNCSHGGTCQD
LVNGFKCVCPPQWTGKTCQLDANECEAKPCVNAKSCKNLIASYYC
DCLPGWMGQNCDININDCLGQCQNDASCRDLVNGYRCICPPGYAG
DHCERDIDECASNPCLNGGHCQNEINRFQCLCPTGFSGNLCQLDI
DYCEPNPCQNGAQCYNRASDYFCKCPEDYEGKNCSHLKDHCRTTP
CEVIDSCTVAMASNDTPEGVRYISSNVCGPHGKCKSQSGGKFTCD
CNKGFTGTYCHENINDCESNPCRNGGTCIDGVNSYKCICSDGWEG
AYCETNINDCSQNPCHNGGTCRDLVNDFYCDCKNGWKGKTCHSRD
SQCDEATCNNGGTCYDEGDAFKCMCPGGWEGTTCNIARNSSCLPN
PCHNGGTCVVNGESFTCVCKEGWEGPICAQNTNDCSPHPCYNSGT
CVDGDNWYRCECAPGFAGPDCRININECQSSPCAFGATCVDEING
YRCVCPPGHSGAKCQEVSGRPCITMGSVIPDGAKWDDDCNTCQCL
NGRIACSKVWCGPRPCLLHKGHSECPSGQSCIPILDDQCFVHPCT
GVGECRSSSLQPVKTKCTSDSYYQDNCANITFTFNKEMMSPGLTT
EHICSELRNLNILKNVSAEYSIYIACEPSPSANNEIHVAISAEDI
RDDGNPIKEITDKIIDLVSKRDGNSIEGRMDPKSCDKTHTCPPCP
APELLGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALPAPIEKTISKAKGQPREPQVYTLPPSRDELTKNQVSL
TCLVKGFYPSDIAVEWESNGQPENNYKTTPPVLDSDGSFFLYSKL
TVDKSRWQQGNVFSCSVMHEALHNHYTQKSLSLSPGK.
